(19) United States
(12) Patent Application Publication
(10) Pub. No.: US 2010/0287638 A1
(43) Pub. Date: Nov. 11, 2010

(54) NEURAL TUMOR STEM CELLS AND METHODS OF USE THEREOF

(75) Inventors: Peter B. Dirks, Toronto (CA); Austin Smith, Great Shelford (GB); Ian D.N. Clarke, Toronto (CA); Steve Pollard, Cambridge (GB)

Correspondence Address: CLARK & ELBING LLP, 101 FEDERAL STREET, BOSTON, MA, 02110, US

(73) Assignees: The Hospital for Sick Children, Toronto (CA); The University Court of the University of Edinburgh, Edinburgh (GB)

(21) Appl. No.: 12/681,036

(22) PCT Filed: Oct. 1, 2008
(86) PCT No.: PCT/CA2008/001741

§ 371 Date: Jul. 26, 2010

Related U.S. Application Data
(60) Provisional application No. 60/997,136, filed on Oct. 1, 2007.
(60) Provisional application No. 61/127,404, filed on May 13, 2008.
(60) Provisional application No. 61/076,119, filed on Jun. 26, 2008.

(51) Int. Cl. C12Q 1/02; C12N 5/09; C40B 30/06; A01K 67/00

(52) U.S. Cl. 800/21; 435/368; 435/29; 506/10

(57) ABSTRACT

The present invention relates to the discovery that renewable stem cell lines can be derived from tumor cells and can be cultured in vitro. Accordingly, the invention provides neural tumor stem cell lines and cells from such cell lines. Because the cell lines retain characteristics of the tumors from which they are derived, the cells can be used in screening methods for identification of potential therapeutic agents and can be used to identify genetic markers which may be predictive for development of such tumors. Finally, such cells can be used to determine an appropriate therapeutic regimen for a patient suffering from a brain tumor. Cells from a patient's brain tumor can be cultured as described herein to create a cell line, and the relative effectiveness of a therapeutic agent against the cells can be tested to determine which agent or combination of agents is most effective in treating the patient's tumor.

### BACKGROUND OF THE INVENTION

The invention relates to neural tumor stem cells and methods of making and using the neural tumor stem cells.

The most common and aggressive type of primary adult brain cancer is malignant glioma. Current treatments for these types of cancers are largely ineffective. Gliomas are classified as astrocytoma, oligodendroglioma, or ependymoma, based on the glial cell type that predominates in the tumor (Kleihues et al., (2000) Pathology and Genetics: Tumors of the Nervous System, 2nd Edition edn: IARC Press, Lyon). Glioblastoma multiforme (GBM) is the most common and aggressive form of malignant astrocytoma, and can arise de novo, or from pre-existing lower grade tumors (Kleihues et al., supra). Individual GBM tumors contain varying proportions of apparently differentiated cell types, alongside ill-defined anaplastic cells. This complicates accurate diagnosis, grading, and sub-classification of the disease. Molecular profiling has suggested distinct molecular classes of disease (Louis, (2006) Ann Rev Pathol 1, 97-117; Mischel et al., (2003) Oncogene 22, 2361-2373). While there has been success in identifying the disrupted signaling pathways and underlying genetic defects associated with glial tumors (Furnari et al., (2007) Genes Dev 21, 2683-2710), it remains unclear how these operate in different cellular contexts.

It is possible that the cellular heterogeneity within each tumor arises from cells that display stem cell characteristics—namely, long-term self-renewal and a capacity to differentiate, as previously demonstrated for leukemia (Lapidot et al., (1994) Nature 367, 645-648). Such cells would underlie a cellular hierarchy, reminiscent of tissue stem cells, and drive tumor growth through sustained self-renewal. The immature cells within GBM express neural progenitor markers such as Nestin (Dahlstrand et al., (1992) Cancer Res 52, 5334-5341). A subpopulation of putative cancer stem cells can be isolated from diverse adult and childhood brain tumors using the neural stem cell marker CD 133 (Hemmati et al., (2003) Proc Natl Acad Sci USA 100, 15178-15183; Singh et al., (2003) Cancer Res 63, 5821-5828), and these can initiate tumor formation following xenotransplantation (Singh et al., (2004) Nature 432, 396-401). These data together with similar approaches for other solid tumors provide support for the cancer stem cell hypothesis (Reya et al., (2001) Nature 414, 105-111; Ward et al., (2007) Annual Rev Pathol 2, 175-189). Despite the desire to obtain glioma neural cancer cell lines, prior to the present invention, the purification and propagation of these cells in vitro has not been successfully achieved. Prior attempts to culture glioma neural cancer cell lines have resulted in the formation of spheres. The use of cellular spheres has several limitations, including fusion, heterogeneity, and progenitor problems.

Accordingly, there is a need for neural tumor stem cell lines, as well as methods for the purification and use of such cells.

### SUMMARY OF THE INVENTION

The present invention relates to the discovery that renewable stem cell lines can be derived from tumor cells and cultured in vitro. These cells remain in an undifferentiated state, but are capable of differentiating into various neural cell types. Accordingly, the invention provides neural tumor stem cell lines and cells from such cell lines. Because the cell lines retain characteristics of the tumors from which they are derived, the cells can be used in screening methods for identification of potential therapeutic agents and can be used to identify genetic markers which may be predictive for development of such tumors. Finally, such cells can be used to determine an appropriate therapeutic regimen for a patient suffering from a brain tumor. Cells from a patient's brain tumor can be cultured as described herein to create a cell line, and the relative effectiveness of a therapeutic agent against the cells can be tested to determine which agent or combination of agents is most effective in treating the patient's tumor.

In a first aspect, the invention features a neural tumor stem cell which expresses at least one (e.g., 2, 3, 4, 5, or 6) of the proteins selected from the group consisting of nestin, Sox2, vimentin, CD44, CD 15, CD 133, GFAP, GFAPδ, and NG2 and has the ability to propagate in an in vitro culture. The tumor may be a glioblastoma multiforme, giant cell glioblastoma, astrocytoma, oligodendroglioma, ependymoma, or medulloblastoma. The cell may be capable of differentiating into neural cell types. The cell may be capable of inducing tumor formation when implanted into the brain of an animal. In certain embodiments, the cell can be propagated in culture for at least 5 (e.g., 10, 15, 20, 35, 50, 75, 100, 200, or 500) passages, or alternatively, can be maintained in culture for at least 1 month (e.g., 2, 3, 4, 5, 6, 8, 10, 12, 15, 18, 24, 36, 48, 60, 90, or 120 months). In certain embodiments, the cells express Sox2, Nestin, CD44, and CD15. The cell may be a human cell.

The invention also provides cells and populations of cells from neural tumor cell lines. Cell lines of the invention include G144-NS (ATCC Deposit No. PTA-8895), G166-NS, G174-NS, G179-NS (ATCC Deposit No. PTA-8894), GliNS1, GliNS2, and EP253-NS.

In another aspect, the invention features a method of producing a neural tumor stem cell line. The method includes the steps of (a) providing a neural tumor sample; (b) culturing cells from the tumor sample under conditions which induce formation of neural cell spheres; (c) dissociating cells from the spheres; (d) applying the cells of step (c) to a substrate under conditions which allow adherence of the cells; and (e) culturing the cells of step (d), thereby generating a neural tumor stem cell line. In certain embodiments, the substrate is charge-modified polystyrene (e.g., poly-L-ornithine/laminin treated polystyrene). The invention also features a neural tumor cell line produced by the method of the invention (e.g., using any of the method steps described herein).

In another aspect, the invention features a method of identifying a candidate compound for the treatment of a neural tumor. The method includes the steps of (a) contacting a neural tumor stem cell capable of undergoing proliferation with a compound; and (b) measuring cellular proliferation of the tumor stem cell following treatment with the compound, where a compound that reduces (e.g., by at least 2%, 5%, 10%, 20%, 30%, 40%, 50%, 60%, 70%, 80%, 90%, 95%, or 99%) proliferation of the cell, as compared to in the absence of the compound, is identified as a candidate compound for the treatment of a neural tumor. In certain embodiments, the candidate compound is selected from a chemical library. The screen may be carried out using high-throughput techniques (e.g., where the cells are in a multi-well plate). The screen alternatively may be carried out in non-human mammal (e.g., a mouse or rat) in which the neural tumor stem cell has been transplanted. In certain embodiments, the cell may be selected from a cell line selected from the group consisting of G144-NS, G166-NS, G174-NS, G179-NS, GliNS1, GliNS2, and EP253-NS.

The invention also features an animal (e.g., a rodent such as a rat or mouse) model of a neural tumor using the neural stem cells (e.g., human) of the invention and a method of making such animals. The method includes the steps of (a) providing at least one neural stem tumor cell, and (b) transplanting the at least one cell into a nervous tissue of a recipient animal. The cell may be from a cell line selected from the group consisting of G144-NS (ATCC Deposit No. PTA-8895), G166-NS, G174-NS, G179-NS (ATCC Deposit No. PTA-8894), GliNS1, GliNS2, and EP253-NS.

In another aspect, the invention features a method for determining whether to administer a compound (e.g., a therapeutic agent) to a patient having a neural tumor. The method including the steps of (a) providing a neural tumor cell from the patient; (b) culturing the tumor cell under conditions sufficient generate a neural tumor stem cell line from the cell; (c) contacting a cell from the cell line with the therapeutic agent; and (d) measuring the proliferation of the cell, wherein a therapeutic agent that reduce proliferation of the cell is identified as a potential therapeutic agent for the patient. The contacting step (c) may further include contacting a second therapeutic agent (e.g., 5, 10, or more). The method may use any compound or therapeutic agent known in the art.

In another aspect, the invention features a method for determining whether to administer a compound to a patient having a neural tumor, said method comprising the steps of (a) providing a cell from neural tumor stem cell line, wherein said stem cell line is derived from a neural tumor cell cultured under conditions sufficient to generate said cell line; (b) contacting said cell from said cell line with said compound; and (c) measuring the proliferation or viability of said cell, wherein a therapeutic agent that reduces proliferation or viability of said cell is identified as a potential therapeutic agent for said patient. The method may further include contacting an additional compound (e.g., 5, 10, 100, 1,000, 10,000 compounds).

In either of the above aspects, the method neural tumor cell or is from a human. The compound may be from a chemical library. The compound may be a chemotherapeutic agent.

By “neural tumor stem cell” is meant a stem cell derived from a neural tumor (e.g., a glioma or any tumor described herein) or a descendent of such a cell that is capable of self-renewal and propagation in culture in an undifferentiated state.

By a “population of cells” is meant a collection of at least ten cells. The population may consist of at least twenty cells, at least one hundred cells, and at least one thousand, or even one million cells. Because the neural tumor stem cells of the present invention exhibit a capacity for self-renewal, they can be expanded in culture to produce populations of even billions of cells. A population of cells may include at least 1%, 5%, 10%, 20%, 30%, 40%, 50%, 60%, 70%, 80%, 90%, 95%, or 99% of a particular cell type (e.g., a neural tumor stem cell).

By “isolated,” in the context of a cell, is meant a cell which has either been isolated from heterologous cells or has been enriched in a population of cells such that the fraction of cells of the desired cell type (e.g., neural tumor stem cells) are in greater proportion than found in nature, e.g., in the organism from which it is derived. For example, a cell may be enriched by 10%, 20%, 50%, 100%, 200%, 500%, 1000%, 10,000% as compared to its proportion in a naturally occurring tissue (e.g., a brain tumor).

By “proliferation” is meant the rate at which cell number increases. A decrease in proliferation may be caused either by an increase in the rate of cell death (e.g., necrotic or apoptotic death), or may be caused by a reduction in the rate of cell division. A decrease in proliferation, caused, for example, by administration of a therapeutic agent to a cell, may be at least 2%, 5%, 10%, 20%, 30%, 40%, 50%, 60%, 70%, 80%, 90%, 95%, 98%, or 99% as compared in the absence of the therapeutic. Rates of proliferation can be measured using any method known in the art (e.g., those described herein).

A “patient” or “subject” can be either a human or a non-human mammal.

Other features and advantages of the invention will be apparent from the following Detailed Description, the drawings, and the claims.

### BRIEF DESCRIPTION OF THE DRAWINGS

FIG. 1 is a set of photomicrographs of glioma neuronal stem (GNS) cells grown on laminin for 1 and 7 days (top, left, and right panels, respectively) and grown in suspension for 1 and 7 days (bottom, left, and right panels, respectively).

FIGS. 2A-2C are photomicrographs of the GliNS2 cell line grown on laminin following direct plating and expansion (2A and 2C, respectively) or neurosphere formation (2B).

FIG. 2D is a photomicrograph of the GliNS2 cell line grown on gelatin.

FIG. 3A is a set of immunophotomicrographs showing the expression of nestin, Sox2, vimentin, and CD44 in four different GNS cell lines.

FIG. 3B is the FACS data for the expression of CD15, CD44, and CD133 in three different GNS cell lines during proliferation (undifferentiated, left and middle columns) and differentiation (right column) of three GNS cell lines.

FIG. 4A shows immunophotomicrographs of the expression of astrocyte (GFAP and GFAPδ), adult neural stem cell (nestin and NG2), and oligodendrocyte precursor markers (Sox10) in three different GNS cell lines.

FIG. 4B is a graph of the fold—increase in the expression of GFAP, GFAPδ, Olig2, PDGFRα, and PDGFα following culture of the G144-NS, G166-NS, G179-NS, GliNS1, CB541, CD192, CB660, fetal, and human ES cells under proliferating conditions in vitro.

FIG. 4C is a Western blot of the expression of GFAPδ, GFAP, and α-tubulin in the G144-NS, G166-NS, G149-NS, GliNS1, and CB541 cell lines.

FIG. 5A is a set of photomicrographs and immunophotomicrographs of cultures of G144-NS, G166-NS, G179-NS, and CB541 cells grown in the presence of EGF and FGF-2: left column shows live cells and right column shows cells immunostained for O4 and TuJ-1 expression.

FIG. 5B is a set of photomicrographs and immunophotomicrographs of G144-NS, G166-NS, G179-NS, and CB541 cells grown in the absence of EGF and FGF-2 for one week (left three columns) and cells grown in the presence of BMP-4 for 5 days (right column).

FIG. 6A is a photograph of the tumor mass resulting from the transplantation of GliNS1 cells into a NOD/SCID mouse.

FIG. 6B is a photomicrograph of a sectioned and stained (hemotoxylin and eosin) mouse tumor resulting from the transplantation of G144-NS cells.

FIG. 6C is a graph showing the FACS data for the expression of CD133 in: (1) a mouse tumor following the transplantation of G144-NS cells; (2) G144-NS cells; and (3) an uncultured xenograft.

FIG. 6D is an immunomicrograph of a sectioned mouse tumor resulting from the transplantation of G144-NS cells, showing the expression of nestin and GFAP.

FIG. 7 is a table showing the frequency of: (1) tumor formation and GNS cell infiltration (dark dot); (2) GNS cell engraftment but not tumor formation (dark grey); and (3) no NGS cells detected in a mouse brain following transplantation of 10⁵, 10³, or 10² NGS cells (G144-NS, G179-NS, G166-NS, G174-NS, GliNS1, and GliNS2 cells shown).

FIG. 8A is set of photomicrographs of a sectioned and stained mouse brain following orthotopic xenotransplantation of G144-NS cells (10,000), G166-NS cells (100,000), G174-NS cells (100 cells), G179-NS (100,000 cells) and fetal neuron stem cells (100,000 cells): left column, unstained; center column, human nestin- and DAPI-stained; and left column, higher magnification of area indicated in corresponding center column.

FIG. 8B is a set of photomicrographs of a sectioned mouse brain following transplantation of G144-NS cells (left panel, primary tumor), transplantation of the primary tumor (center panel, secondary tumor), and transplantation of a secondary tumor (right panel, tertiary tumor).

FIG. 9 is a set of photomicrographs of a sectioned and human nestin-stained mouse brain 5 weeks after transplantation of GNS cells.

FIG. 10 is a picture and set of photomicrographs of a mouse tumor resulting from transplantation of G166-NS cells.

FIG. 11 is a set of photomicrographs of sectioned mouse brains from a control animal (left column) and from animals receiving transplanted G174-NS (second to left column), G144-NS (second to right column), and G166-NS cells (right column).

FIGS. 12A and 12B are photomicrographs showing derivation and initial characterization of GNS cells. FIG. 12A shows a representative example of primary cultures established by plating of glioma tumor populations directly on a laminin substrate in NS cell expansion media (left panel) or parallel cultures initially grown in suspension as neurospheres and then re-plated and allowed to attach to a fresh laminin-coated flask (right panel). FIG. 12B shows NS cell markers in GNS cells. Immunocytochemistry for the markers Nestin, Sox2, Vimentin, and CD44 in three different glioma cell lines (G144, G166, and G179) and one fetal NS cell line (CB541).

FIGS. 13A-13F show that G144 cells generate tumors following xenotransplantation. FIG. 13A shows GNS cells transplanted into immunocompromised mice, which and resulted in a large tumor mass (22 weeks after transplantation of 10⁵ G144 cells, passage 18). FIG. 13B shows a similar xenograft tumor sectioned and assessed for histopathology. This tumor displayed hallmarks of GBM. FIG. 13C shows quantification of CD 133⁺ cells within the directly harvested/uncultured xenograft compared to the original patient tumor using flow cytometry. FIG. 13D shows immunocytochemistry for Nestin (red) and GFAP (green) in xenograft tumors which confirmed heterogeneity. FIGS. 13E and 13F shows that, while the original patient tumor for G144 was graded as GBM, CNPase⁺ oligodendrocyte-like cells are in fact widespread, consistent with the G144 in vitro differentiation. HE, haemotoxylin and eosin. DAPI nuclear counterstain, blue.

FIGS. 14A and 14B show that all GNS cell lines are tumorigenic. FIG. 14A shows that xenotransplantation of each GNS cell line led to formation of a tumor mass, with highly infiltrative behavior (arrow). Left panels show coronal section of brain stained with H and E. Right panels show staining for human nestin in xenograft tumors (boxed region of middle panels). Fetal NS cells (hf240) fail to generate tumors. FIG. 14B shows that G144 xenograft derived cells serially transplanted into secondary and then tertiary hosts also resulted in tumor formation.

FIGS. 15A-15C show that GNS cell lines exhibit distinct differentiation responses in vitro. FIG. 15A shows that, in proliferating conditions (EGF and FGF-2), there is no detectable differentiation of G144, G166, or G179 cells into oligodendrocytes (green, O4⁺) or neurons (red, TuJ-1⁺) by morphology or immunostaining. FIG. 15B shows that, 7 days following growth factor withdrawal, there is clear differentiation of G144 cells into O4⁺ oligodendrocytes, while G179 seems to make TuJ-1⁺ neurons more readily. FIG. 15C shows that, following exposure to BMP-4 for 1 week, both G144 and G179 efficiently differentiate into GFAP⁺ cells, together with a minor population of Dcx⁺ neuronal precursors. ('Live', phase-contrast image of live cultures). FIG. 15D shows GFAPδ (green) and CNPase (red) immunostaining in the original patient tumor for G179 and G144.

FIGS. 16A-16D show that GNS cells express lineage-specific characteristics. FIG. 16A shows immunostaining of cells grown in proliferating conditions identifies differential expression of lineage markers. FIG. 16B shows quantitative RT-PCR for lineage markers. FIG. 16C shows an immunoblot for the adult SVZ astrocyte marker GFAPδ. Control (hES) is a human embryonic stem cell line (hED1). FIG. 16D shows GFAPδ expressing cells were also identified in the original G179 patient tumor. The original G144 patient tumor contains large numbers of CNPase cells and cells with lower levels of GFAPδ.

FIGS. 17A and 17B show that GNS cells are more similar to fetal NS cells but have distinct phenotypes. FIG. 17A shows principal component analysis (PCA) of global mRNA expression in each GNS cell (black, G144, G144ED, G166, G179, G174, and GliNS2), fetal NS cells (red, hf240, hf286, and hf289), and normal adult brain tissue (blue). ‘a’ and ‘b’ signify biological replicates. FIG. 17B shows hierarchical clustering of a set of established NS cell markers, lineage markers, and known glioma ‘tumor pathway’ genes. (*OLP-expressed genes; **GFA Pδ-specific probe).

FIGS. 18A-18D shows that GNS cells are suitable for cell imaging-based drug screens. Effects on cell proliferation following addition of a library of 450 compounds to GNS cells are shown. FIG. 18A shows relative cell number, derived from quantitative analyses of microphotographs plotted against time for an example plate of the G179 screen. In red are all the “hits,” compounds acting within the lowest 5* percentile, reducing cell number to <0.75. Every tenth well for all other compounds are plotted in blue to illustrate the distribution range. The Z-factor for this screen was 0.76 (see methods described below). The dotted blue line refers to Tryptoline. FIG. 18B shows a cartoon of an example 96-well plate for GNS cell line G179, and HS27 (fibroblasts). Indatraline (label *) and Paroxetine (label **) show differential effects affected all GNS cells but not fibroblasts (HS27). Confluence readings after 2 days clearly identify cytotoxic drugs. FIG. 18C shows Summary of active compounds: red indicates compounds identified within the 5* percentile of cell confluence in two out of two screens, orange in one of two screens, blue in neither of the two. FIG. 18D shows validation of the results of the screen. Here, selected compounds from an independent source were applied (2 μM for Tegaserod, 10 μM for all others) to G179 (left), HS 27 (middle) or fetal NS (right). Live images after 2 days of treatment with each compound are shown.

FIG. 18E is a set of photomicrographs of cells either untreated or treated with indatraline and stained for TUNEL and caspase. The indatraline treated cells show an increase in TUNEL and caspase staining, thus indicating that indatraline causes cell death via an apoptotic pathway.

FIG. 19A shows molecular cytogenetic analysis of G144. Shown are the SKY and FISH findings for Early and Late Cultures of G144. For each passage, the inverted DAPI, Red-Green-Blue (RGB), and classified karyotype is shown. In the early passage G144, both diploid, but predominantly tetraploid populations (shown) were detected. No gross structural rearrangements were detected. In the late passage G144, a significant change in ploidy was identified resulting in a predominantly pentaploid genome with the net gains of chromosome 7. Structural rearrangements were identified as shown by the change in color along the length of a contiguous chromosome. To confirm the net copy-number gains of chromosome 7, FISH using a centromere probe for chromosome 7 (green) and the EGFR locus (red) was performed on both cell lines as well as the formalin-fixed paraffin embedded original patient specimen. FISH to the original patient specimen identified on average, 3 copies of chromosome 7 per cell, consistent with the net gain of chromosome 7 in the diploid population of the early G144 culture, suggesting the early passage maintained some similarity to the original specimen.

FIG. 19B shows molecular cytogenetic analysis of G179. Shown are the SKY and FISH findings for Early and Late Cultures of G179. For each passage, the inverted DAPI, Red-Green-Blue (RGB) and classified karyotype is shown. Both early and late passages were found to maintain overall hypertriploidy as well as the maintenance of structural rearrangements. The early passage showed 6 whole chromosomes 7, while the later passage revealed the loss of one whole chromosome and the presence of a deleted chromosome 7, still containing EGFR. To confirm the net copy-number gains of chromosome 7, FISH using a centromere probe for chromosome 7 (green) and the EGFR locus (red) was performed on both cell lines as well as the formalin-fixed paraffin embedded original patient specimen. FISH to the original patient specimen identified variable copies of chromosome 7 per cell ranging from 4-6 copies, consistent with the net gains of chromosome 7 seen in the early and late cultures.

FIGS. 20A-20D show that, five weeks following xenotransplantation, G144 cells have engrafted and infiltrated the host brain. FIG. 20A shows a coronal section of transplanted adult mouse brain. FIG. 20B shows a boxed region in 20A. FIG. 20C shows immunohistochemistry for human nestin of region shown in 20B (green). FIG. 20D shows that cellular and nuclear pleomorphism is apparent at higher magnification.

FIGS. 21A-21D show that G144 clonal cell lines exhibit heterogeneity in lineagemarkers and can generate oligodendrocytes similar to the parental population. Similar results were seen for two other independent clonal lines. FIG. 21A shows Olig2 immunocytochemistry on proliferating cells. FIG. 21B shows Sox10 and NG2 co-staining in proliferating cells. FIG. 21C shows that or oligodendrocyte-like cells are generated 7 days after growth factor withdrawal. FIG. 21D shows astrocyte-like GFAP⁺ cells are present following 7 days of BMP treatment.

FIGS. 22A-22C show xenograft tumors generated from G144 contain oligodendrocytes similar to the original patient tumor. FIG. 22A shows histopathology of the xenograft tumor shows G144 tumors contain cells with ‘fried egg’ appearance indicative of oligodendrocytes, as does a clonal cell line derived xenograft tumor. FIG. 22B shows that heterogeneity is observed with clonal derived tumors. FIG. 22C shows that nestin-expressing cells are enriched around the periphery of the tumor mass.

FIG. 23 shows GFAP [symbol] and total GFAP immunocytochemistry in proliferating G179 cells. From the overlay (right), GFAP [symbol] filaments localize more to the cell body and peri-nuclear regions.

FIG. 24 shows that GliNS2 also expresses Olig2, NG2 and Sox 10 (left and middle panel) and can generate readily oligodendrocyte upon growth factor withdrawal (right panel).

FIG. 25 shows flow cytometry analysis of GNS cell surface marker expression (CD15, CD44, and CD133) in proliferating conditions and following differentiation (serum exposure for 14 days).

FIG. 26 shows that chromosome 7 and 19q genes are significantly differentially expressed between NS cells and GNS cells. The significantly differentially expressed genes located within these two regions are shown in the heatmap.

FIG. 27 shows a heatmap of markers differentially expressed between GNS cells and foetal NS cells, excluding those expressed on chromosome 7 and 19q.

### DETAILED DESCRIPTION

We have identified a method for producing lines of tumor stem cells from central nervous system tumors and have generated several such cell lines. Accordingly, the present invention provides neural tumor stem cells and cell lines (e.g., glioma stem cell lines, such as those described herein), methods for generating such cell lines, screening methods for identification of therapeutic agents, and methods for determine whether an agent or set of agents will be effective in treating a patient's tumor, as shown in the examples described herein.

### GNS Cell Lines

We have demonstrated that adherent culture methods established for fetal and human NS cells provide a reliable technique for reproducibly isolating cell lines with stem cell and cancer initiating properties from gliomas. Our findings show that suspension culture is not a requirement for successful long-term propagation of tumor-derived stem cells. In fact, by expanding glioma tumor initiating cells as adherent cell lines, some of the limitations of the neurosphere culture paradigm are overcome (Reynolds et al., (2005) Nat Methods 2, 333-336). GNS cells are highly tumorigenic and resulted in tumors that are strikingly similar to the human disease, while retaining patient-specific characteristics.

Human fetal NS cell lines display features also exhibited by gliomas such as immortality, EGFR signaling dependence, and bias towards glial differentiation (Pollard et al., (2006) Cereb Cortex, 16 Suppl 1, i112-i120; Sun et al., (2008) Mol Cell Neurosci 38, 245-258). Thus, the NS cell state in vitro may be sustained by similar mechanisms to those that operate in stem-like cells in glioma. Crucially, however, NS cells expanded in vitro do not generate tumors when transplanted. By contrast, the GNS cell self-renewal program is not extinguished in vivo and cells generate infiltrative tumors that closely resemble the human disease.

CD44 has been used to enrich for putative cancer stem cells in other types of solid cancer such as breast, head and neck, pancreas, and prostate (Al-Hajj et al., (2003) Proc Natl Acad Sci USA 100, 3983-3988); (Li et al., (2007) Cancer Res 67, 1030-1037; Patrawala et al., (2006) Oncogene 25, 1696-1708; Prince et al., (2007) Proc Natl Acad Sci USA 104, 973-978). All GNS cell lines tested here express high levels of CD44, similar to fetal NS cells. Although not a specific marker of stem cells, cell sorting of CD44-expressing cells has proved useful for enrichment of mouse NS cells from diverse progenitor populations, and CD44 expression may mark FGF-responsive subpopulations (Pollard et al., (2008) Mol Cell Neurosci, In press). CD44 has also been characterized in gliomas and may be required for the infiltration of the normal brain that characterizes high-grade gliomas (Bouterf et al., (1997) Neuropathol Appl Neurobiol 23, 373-379). High CD44 expression within brain tumors is associated with poor patient survival (Ranuncolo et al., (2002) J Surg Oncol 79, 30-35; discussion 35-36). It will be of interest to determine in future studies whether differences in levels of CD44 expression serve as a dual marker of glioma cells that exhibit both extensive self-renewal and infiltrative behavior. Our initial findings suggest CD44 can be used for enriching the self-renewing population (SP, unpublished data). As CD44 is expressed by astrocyte-restricted progenitors as well as NS-like cells (Liu et al., (2004) Dev Biol 276, 31-46), it may provide a more general marker of use for enriching tumor initiating cells from lower grade tumors.

Despite broad similarities to fetal NS cells we also find distinct patterns of differentiation and marker expression between GNS cell lines, suggesting that gliomas are not driven by a single phenotypic type of tumor stem cell. In particular, not every line demonstrated expression of CD133, indicating that this marker does not universally identify tumorigenic cells in malignant glioma. Differences in differentiation behavior between tumor neurospheres have been reported previously, and are suggested to be a consequence of the differential expression of BMPR1B (Lee et al., (2008) Cancer Cell 13, 69-80), or misregulation of the dif ferentiation program (Galli et al., (2004) Cancer Res 64, 7011-7021). Gunther et al., recently reported that glioblastoma-derived stem cell cultures fall into two distinct subgroups, based on their adhesion properties (Gunther et al., (2008) Oncogene 27, 2897-2909).

Tumor-specific stem cell states can be distinguished based on lineage specific markers and differentiation behavior. Within the developing and adult nervous system there are many distinct classes of proliferative progenitors (e.g., neuroepithelial cells, radial glia, glial progenitors, oligodendrocyte precursors, and SVZ astrocytes). G144 cells strongly express markers of the oligodendrocyte precursor cell lineage and are biased towards oligodendrocyte differentiation. By contrast, G179 has more similarity to adult SVZ astrocytes, such as expression of GFAPδ and a capacity to generate neurons in vitro (Sanai et al., (2004) Nature 427, 740-744). G166 cells appear quite distinct to each of these and lack expression of CD133. This has also been reported for subsets of glioma-derived neurospheres (Beier et al., (2007) Cancer Res 67, 4010-4015). The wide and continuous histological spectrum of gliomas, with regard to proportions of the various differentiated and anaplastic cells, may therefore be strongly influenced by the phenotype of the underlying tumor initiating cells. If so, detailed characterization of GNS cell lines from larger Glioma NS cell lines numbers of patients and comparison with patient outcome and pathology reports may help in sub-classification of gliomas. It should also now be possible to derive GNS cell lines from previously established glioma neurosphere cultures, in order to more rigorously define the identity and variety of stem cell subtypes.

Further, GNS cells can be genetically modified, enabling additional chemical or genetic screens, e.g., assays of differentiation based on lineage-specific fluorescent reporters, or morphometric analysis of cell behavior. We have also demonstrated their potential utility in assaying cell motility, an important feature of malignant gliomas (Dirks, P. B. (2001) J Neurooncol 53, 203-212). Also, RNAi screens using live time-lapse imaging of human cells have been reported (Neumann et al., (2006) Nat Methods 3, 385-390) and similar technologies could be transferred to GNS cells. Suspension culture methodology is currently being applied to a range of solid tumors, such as breast cancer (Liao et al., (2007) Cancer Res 67, 8131-8138) and colon cancer (Ricci-Vitiani et al., (2007) Nature 445, 111-115). We believe that for other solid tumors, particularly those driven by EGFR signaling, derivation of adherent stem cell lines using similar culture conditions could offer significant advantages.

GNS cells provide a versatile and renewable resource to screen for new drugs. The ability to generate patient-specific tumor NS lines provides an opportunity to test panels of drugs and drug combinations on individual patient tumor lines in vitro, in order to develop patient-tailored treatments. Stem cell self-renewal, migration, apoptosis, and differentiation represent critical therapeutic targets. We demonstrated utility of GNS cells by carrying out a small scale chemical screen of known pharmaceutical drugs. The present screen extends to human brain cancer stem cells our previous observation that mouse neurospheres are sensitive to modulation of neurotransmitter pathways (Diamandis et al., (2007) Nat Chem Biol 3, 268-273).

### Methods for Purification and Propagation of Neural Tumor Stem Cells

The invention provides methods of producing cells lines of neural tumor stem cells. The method is generally applicable to any central nervous system tumor. Indeed, neural tumor stem cells lines from glioblastoma multiforme (GBM; WHO grade IV astrocytomas); mixed oligodendrocyte/astrocyte tumors; ependymomas (4 separate lines); and medulloblastomas have been generated.

Following obtaining tumor tissue from a patient, proliferating tumor cells were grown as neurospheres as previously described (Singh et al., Cancer Res. 63:5821-582815, 2003, and Singh et al., Nature, 432:396-401, 2004). Briefly, tumors were washed, acutely dissociated in oxygenated artificial cerebrospinal fluid and subject to enzymatic dissociation as described previously (Reynolds et al., Science 255:1707-1710, 1992). In one example, the tumors were minced into small pieces (<1 mm) in buffer. Artificial cerebral spinal fluid (ACSF) was used in most cases, and although not essential, this buffer resulted in better viability than buffers such as PBS or Hanks Balanced Salt Solution. The tumors were then digested for 30-90 minutes at 37° C. in ACSF supplemented with trypsin (1.33 mg/ml), hyaluronidase (0.67 mg/ml), and kynurenic acid (0.1-0.17 mg/ml), or just until you can break the tumor apart into single cells. The time required for this step varied from tumor to tumor. Any method for dissociating cells known in the art may be used in the methods of the invention.

The cells were collected by centrifugation and resuspended in 2 ml human neural stem cell (hNSC) media (1×DMEM:F12 (plus antibiotics), 1×N2 Supplement (available from Invitrogen), 20 ng/ml EGF (human recombinant, Sigma), 20 ng/ml bFGF (Upstate), 2 mg/ml heparin, 10 ng/ml LIF (Chemicon), 1×NSF-1 (Clonetics), and 60 μg/ml N-acetylcysteine (Sigma). The cells were then triturated to break up clumps and dissociated into single cells and filtered through a cell strainer. Red blood cells, if present, can be removed using Lympholyte gradient (Cedarlane Laboratories product).

In one embodiment, the cells were then placed into tumor sphere media at 1−2×10⁵ cells/cm² (see Singh et al., Cancer Res, supra and Singh et al., Nature, supra). The tumor sphere media (TSM) consists of a chemically defined serum-free neural stem cell medium (Reynolds et al., Science 255:1707-1710, 1992), human recombinant EGF (20 ng/ml; Sigma), bFGF (20 ng/ml; Upstate), leukemia inhibitory factor (10 ng/ml; Chemicon), Neural Survival Factor (NSF) (1×; Clonetics), and N-acetylcysteine (60 μg/ml; Sigma; Uchida et al., Proc. Natl. Acad. Sci. USA, 97:15720-15725, 2000). The cells were plated at a density of 3×10⁶ live cells/60-mm plate.

The cells that attached to the plastic dish and did not proliferate were removed and not used for deriving the tumor NS cells, as these cells. This step, while likely not essential for culturing high-grade tumors, speeds the process of culturing. Accordingly, this step is more important when culturing lower grade tumors that have very few proliferating cells.

Culturing the cells under these conditions resulted in spheres forming. Depending on how fast the tumor grows, sphere formation typically required 3-5 days. The spheres were then removed and dissociated with 3-5 minute digestion with Accutase™ (Sigma-Aldrich Chemicals), although other dissociation methods such as trypsin may be used. These cells were then plated onto modified Poly-L-Ornithine/Laminin dishes in NS media which includes Neurocult™ NS-A Basal medium (Human) (Stem Cell Technologies, Vancouver, Canada); 2 mM L-Glutamine; 1× Antibiotic/antimycotic; 1× Hormone mix (equivalent to N2 serum free supplement, which is commercially available); 1× B27 supplement (Invitrogen); 75 μg/ml BSA; 10 ng/ml recombinant human EGF; 10 ng/ml bFGF; and 2 μg/mlHeparin. The plates used for this step were generated as described below.

While some stem cells lines stick well to regular plastic tissue culture dishes, most do not. Consistent attachment and growth was observed only with specially charge modified polystyrene dishes designed for high attachment of these cells. We generated our own plates from commercially available plates (Falcon-Primaria™ from BD Biosciences and CellBind™ from Corning), which were sequentially treated with poly-L-ornithine and laminin for increased attachment and growth as follows. A 0.01% solution of poly-L-ornithine (Sigma, Cat #P4957) was added to plates and flasks for at least 20 minutes. The solution was removed and plates/flasks were washed with 1×PBS. The PBS solution was replaced with a 5 μl/ml solution of laminin in PBS (Sigma, Cat: L2020) and the plates were incubated at 37° C. for at least 3 hrs (preferably overnight) to generate the modified plates.

The cells usually attach rapidly to modified plates but may take several days to become consistently adherent. The were split by treating with Accutase™ (Sigma) until detached (3-5 min) and passaged 1:2 or 1:3 onto fresh plates or dishes in NS media. Optimal cultures are maintained by keeping the cells from getting too dense (<70% confluence). Rates of cell line growth can vary, but it typically requires 6-12 weeks to establish a line.

### Cells and Cell Lines

The neural tumor stem cells of the invention can be purified and proprogated by the any of the methods described herein. The stem cells can express one or more (e.g., at least 2, 3, 4, 5, or 6) of the following cellular markers: CD44, CD133, CD15, nestin, vimentin, and Sox2. The glioma neural stem cell can express any combination of markers, for example: CD44 and one or more of CD133, CD15, nestin, vimentin, and Sox2; CD133 and one or more of CD44, CD15, nestin, vimentin, and Sox2; or CD15 and one or more of CD44, CD133, nestin, vimentin, and Sox2. In one example, the cells express Sox2, Nestin, CD44, and CD15.

In addition to the expression of these markers, the neural tumor stem cell lines of the invention can also maintain the ability to differentiate (e.g., into neural cell types) following prolonged culture (e.g., at least one, two, or three week; at least 1, 2, 3, 4, 5, 6, 7, 8, 9, 10, 11, or 12 months, or at least 1, 2, 3, 5, 7, or 10 years) in vitro. Stated in different terms, the glioma neural stem cell lines may maintain the ability to differentiate following at least 2, 4, 6, 9, 10, 12, 15, 20, 25, 30, 40, or 50 passages.

In addition to the expression of the above cellular markers, the neural tumor stem cells may also have the ability to maintain in an undifferentiated state following prolonged culture in vitro (e.g., at least 1, 2, or 3 weeks; at least 1, 2, 3, 4, 5, 6, 7, 8, 9, 10, 11, or 12 months, or at least 1, 2, 3, 5, 7, or 10 years).

In general, the neural stem cells of the invention retain cancer stem cell characteristics and, further, retain characteristics of the original tumors from which they are derived. One characteristic common to all cells is that they are very dynamic. Observation of the cells using time-lapse video microscopy in culture has shown that all neural tumor stem cells change shape rapidly and move around on the substrate. This is unusual, but is very similar to non-tumor neural stem cells. For example, the cells can appear as small rounded cells and, within five minutes, have flat elongated bipolar shape or polygonal with many cellular processes. Each cell line has its own general characteristics (G144-NS, for example, is small with fewer processes, whereas G179-NS is large with mostly bipolar characteristics).

One common feature of these cells is the ability to differentiate into multiple lineages of CNS cells. They retain this ability after more than 36 months continuously in culture. The cells generate various types of cells including astrocytes, oligodendrocytes, and neurons upon growth factor withdrawal. The types of cells and ratio of various lineages can change depending on the procedure used to differentiate the cell lines, and these characteristics again vary between different cell lines.

The cells can accumulate some cytogenetic changes as would be expected from tumor cells, but they typically do not have major chromosomal rearrangements. They can acquire anuploidy changes. Each glioma cell line has a defined character when transplanted into immunodeficient mice. The different neural tumor stem cell lines give reproducible and distinct types of tumors in these mice.

In addition to the expression of the above cellular markers, the neural tumor stem cells may also have the ability to induce a neural tumor in a model animal following xenotransplation.

The neural tumor stem cells of the invention may have one or more of any of the activities listed above.

### Screening Methods

The cell lines described herein or generated using the methods of the invention are useful in screening for candidate compounds for treatment of neural tumors such as glioblastoma multiforme, giant cell glioblastoma, anaplastic oligodendroglioma, ependyoma, and medulloblastoma. In vitro screening assays or assays involving screening of animals having received transplanted neural tumor stem cells can be used to identify potential therapeutic compounds which decrease proliferation tumor stem cells.

Screening assays to identify compounds that decrease cell proliferation (e.g., by reducing the rate of cellular division or by increasing cell death through, for example, necrotic or apoptotic mechanism) are carried out by standard methods. The screening methods may involve high-throughput techniques.

Any number of methods is available for carrying out such screening assays. In one approach, candidate compounds are added at varying concentrations to the culture medium of neural tumor stem cells. Rates of cell proliferation can be measured using any method known in the art; the precise method is not critical to the invention. Rates of cell growth can be measured by cell counting, or by measuring incorporation of labeled nucleotide analog such as BrdU. Alternatively, cell viability can be measured using a vital dye, such as Alamar Blue. Markers for apoptotic death, can be used as well, e.g., antibodies for protein markers such as caspases and bcl, or markers for other cellular changes such as DNA fragmentation using TUNEL labeling. A compound that promotes a decrease in cell proliferation is considered useful in the invention; such a molecule may be used, for example, as a therapeutic for a treating a neural tumor (e.g., a glioma).

### Test Compounds and Extracts

In general, compounds capable of treating a neural tumor (e.g., a glioma) are identified from large libraries of natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating metabolic disorders should be employed whenever possible.

When a crude extract is found to have an activity that inhibits proliferation of a tumor stem cell line, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating a neural tumor (e.g., a glioma). Methods of fractionation and purification of such heterogenous extracts are known in the art. If desired, compounds shown to be useful agents for the treatment of a neural tumor (e.g., a glioma) are chemically modified according to methods known in the art.

### Customized Therapy

The present invention also provides methods for identifying a treatment course for a particular patient having a neural tumor, based on screening of cells taking from the patient's tumor. Briefly, these methods involve taking tumor cells from patient, culturing the tumor cells (e.g., as described above) to generate a tumor stem cell line, and contacting the tumor stem cells with a therapeutic agent or combination of therapeutic agents and measuring cellular proliferation (e.g., as described herein). An agent or combination of agents which reduces cellular proliferation in vitro (e.g., by reducing the diis thus identified as a potential therapeutic agent or combination of agents for use in that particular patient. By comparing the effect of multiple therapeutics against a particular patient's tumor stem cells, optimized therapeutic regimens can be identified. Any of the screening methods described above may be used in determining a customized therapeutic regimen. Any agents (e.g., those known to treat tumors such as Carboplatin (Paraplatin), Carmustine (BCNU, BiCNU), Lomustine (CCNU), Cisplatin (Platinol), Temozolomide (Temodar), and Vincristine (Oncovin or Vincasar PFS); other exemplary agents are described in the examples below) can be used in the methods for identifying a treatment regimen for a patient. In certain embodiments, libraries of compounds (e.g., the NIH Clinical Collection described herein) can be screened against the cells. Screening can be performed using any methods known in the art (e.g., the live screening methods described herein).

### Genetic Marker Analysis

The tumor cell lines of the invention can also be used to identify genetic markers for the propensity to develop a neural tumor. Using differential expression techniques, protein or expression markers for neural cancer can be identified. Once particular genes are identified, genetic analysis to determine whether particular mutations in the coding regions or non-coding regions of the gene. Such changes can include single nucleotide polymorphisms (SNPs), insertions, or deletions. These changes can be analyzed over patient populations to determine if certain changed are correlated with an increased risk of developing a neural tumor (e.g., a glioma or any other tumor described herein). The SNP database available through the National Center for Biotechnology Information (NCBI) website can, for example, be used in the analysis.

### Animals Models

The neural tumor stem cells of the invention may be used to generate animal models of neural tumors. Such methods are known in the art, and include the transplantation of a number of glioma neural stem cells into a model animal such as a rat or mouse. Methods of cell transplantation and immunodeficient recipient animals are described, for example, in U.S. Pat. No. 5,491,284, hereby incorporated by reference. Exemplary transplantation of neural tumor stem cells into animals (e.g., rodents) is described below.

The following examples are meant to illustrate rather than limit the invention.

### EXAMPLES

### Example 1

### Glioma Neural Stem Cells May be Purified from Diverse Gliomas

Neural tumor stem cells were successfully purified according to the methods provided herein, from a number of gliomas (Table 1), including: glioblastoma multiforme (GBM), giant cell glioblastoma multiforme (giant cell GBM), anaplastic oligoastrocytoma, and ependyoma.

TABLE 1
GNS cell lines derived in this study
and corresponding patient details

| NS cell line | final diagnosis | age/gender |
|---|---|---|
| G144-NS | GBM | 51M |
| G166-NS | giant cell GBM | 73F |
| G174-NS | Anaplastic oligoastrocytoma | 60M |
| G179-NS | GBM | 52M |
| G179-NS | GBM | 51M |
| GliNS1 | GBM | 54M |
| EP253-NS | ependymoma | unknown |

### Example 2

### Gns Cell Lines May be Grown on a Substrate or in Suspension

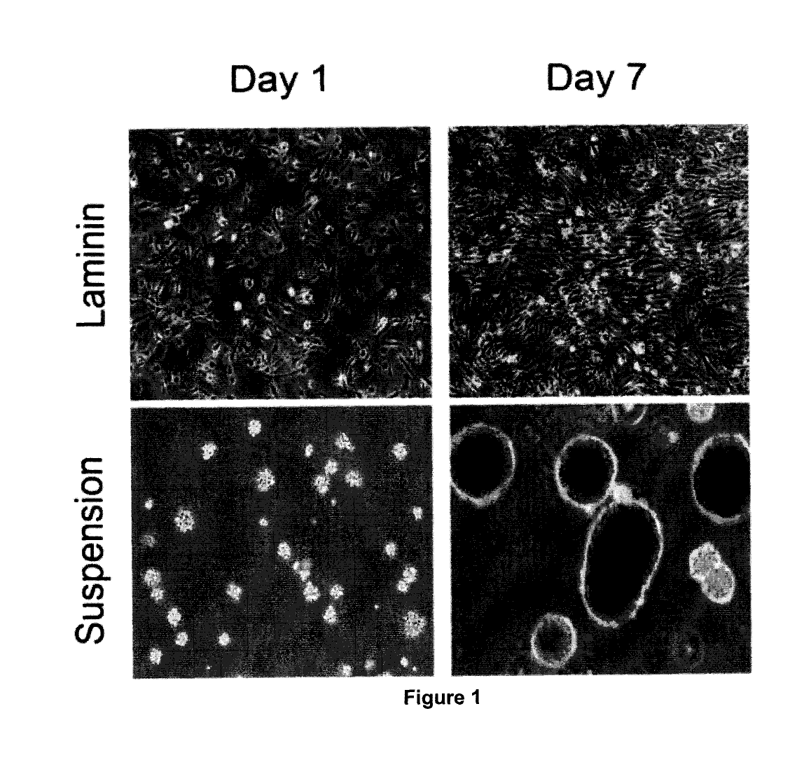

The glioma neural stem cell lines of the invention may be successfully grown in suspension or grown on laminin (FIG. 1). Although better plating efficiency for the glioma neural stem cell lines is observed for laminin, gelatin may also be used as a substrate.

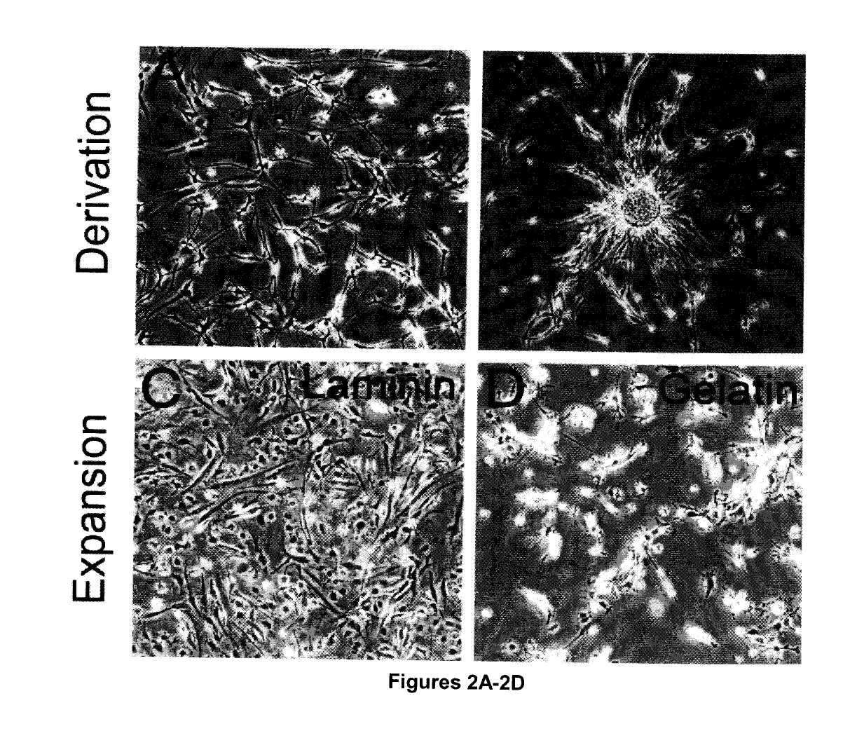

Five GNS cell lines were successfully grown in culture for at least one year (20 passages). For one glioma neural stem cell line, GilsNS2, adherent cultures were derived by direct plating onto laminin or through neurosphere formation followed by attachment and outgrowth on laminin (FIGS. 2A-2C).

### Example 3

### GNS Cells Line Expression of Cellular Markers

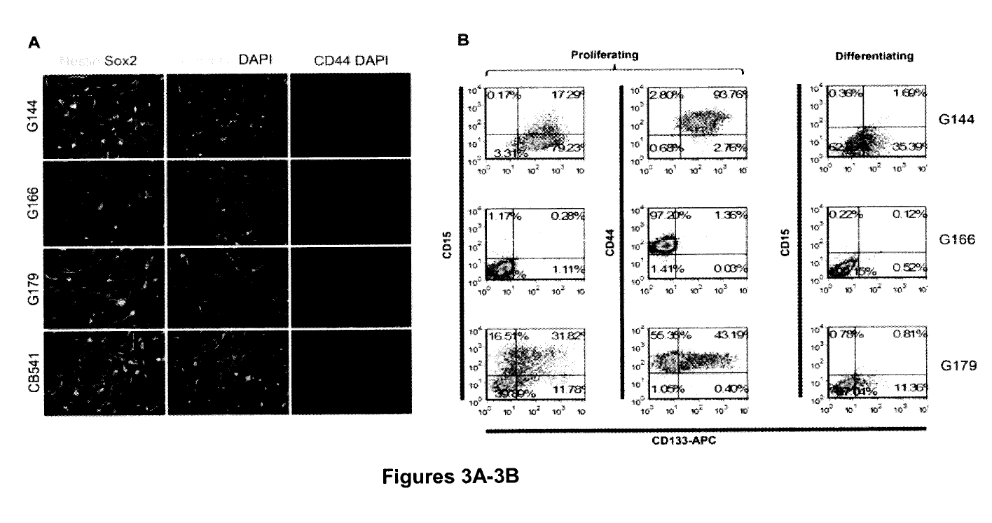

The ability of GNS cells to express different cellular markers of undifferentiated, stem or precursor cells (i.e, CD44, CD133, CD15, nestin, vimentin, Sox2, Olig10, and NG2) was determined by immunocytochemistry. Among four GNS cell lines analyzed, uniform CD144 expression was observed in all the cell lines, however, there was some heterogeneity in the expression of CD15 and CD133 (FIG. 3A). Under differentiating conditions, three of the cell lines show reduced expression of CD15 and CD133 (FIG. 3B).

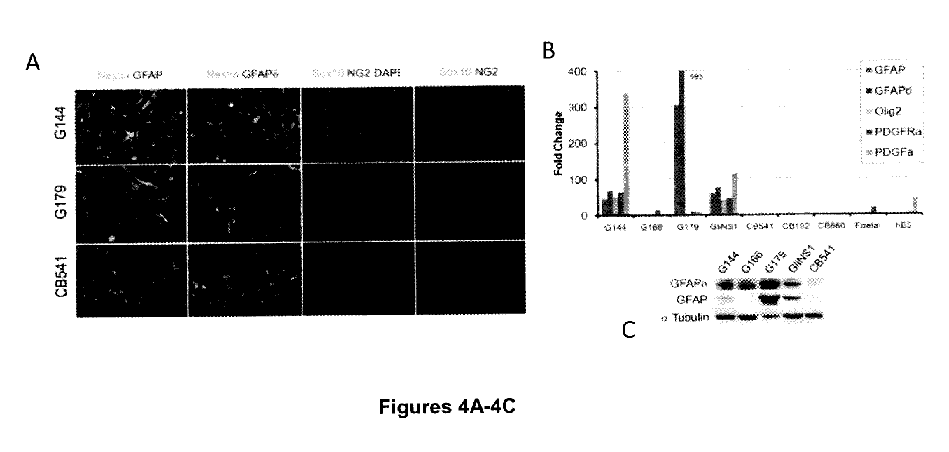

The GNS cell lines also have differences in the expression of astrocyte, adult neural stem cell, and oligodendrocyte precursor markers (FIGS. 4A-4C). Astrocyte precursor markers include GFAP and GFAPδ, adult neural stem cell markers include nestin and NG2, and an example of an oligodendrocyte precursor marker is Sox10. The results indicate that GFAPδ is more highly expressed in the G179-NS cell line than in the G144-NS cell line. The G144-NS cell line expresses the oligodendrocyte precursors Sox10 and NG2.

### Example 4

### GNS Cells Maintain the Ability to Differentiate

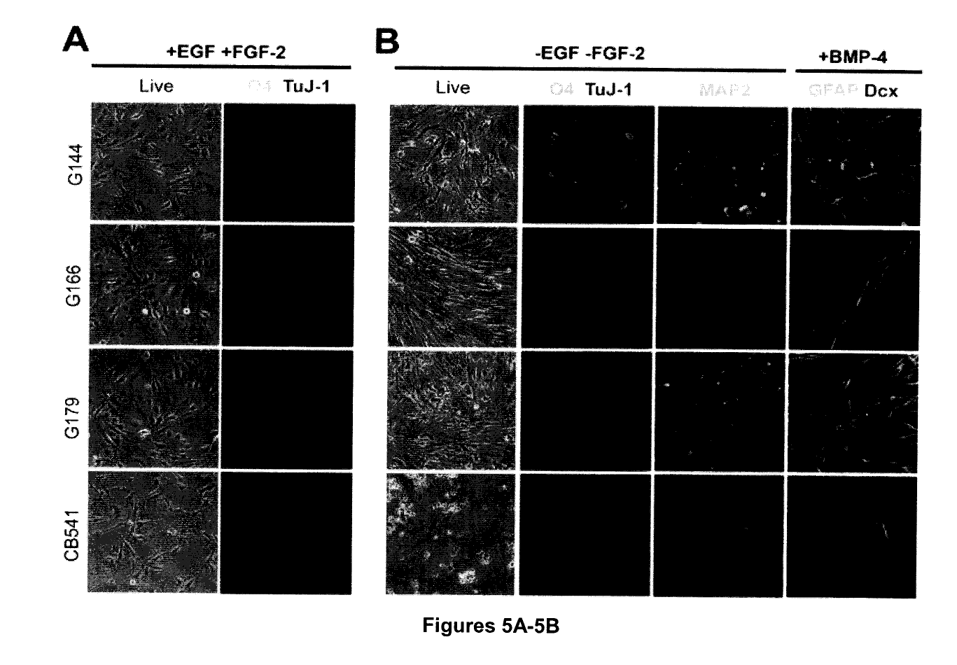

Three different GNS cell lines all show differentiation capacity in vitro following either growth factor withdrawal or treatment with BMP-4 (FIGS. 5A-5B). In proliferating conditions (i.e., in the presence of EGF and FGF-2) there is no differentiation of the cells to oligodendrocytes or neurons. Upon removal of growth factors, oligodendrocyte differentiation occurred within one week for the G144-NS cell line, and MAP2-expressing neuron-like cells appeared at three weeks. For the G179-NS cell line, withdrawal of the growth factors resulted in the formation of neurons (as measured by O4 and TuJ-1 expression). By contrast, G166-NS did not differentiate following withdrawal of growth factors, but did differentiate after BMP-4 treatment. Thus, the GNS cells fulfill the criteria of stem cells as they are long term expandable and retain differentiation capacity.

### Example 5

### GNS Cells are Highly Tumorigenic Following Xenotransplantation

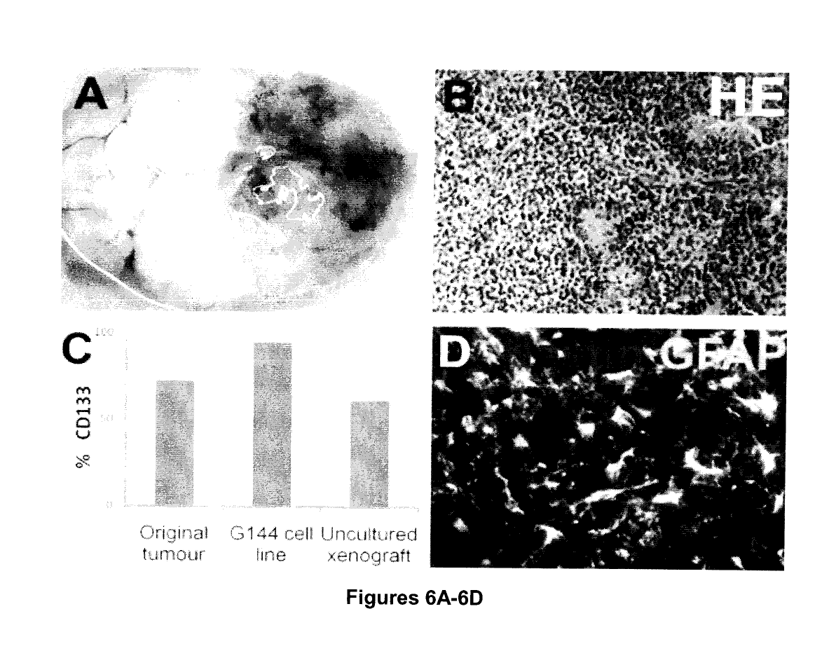

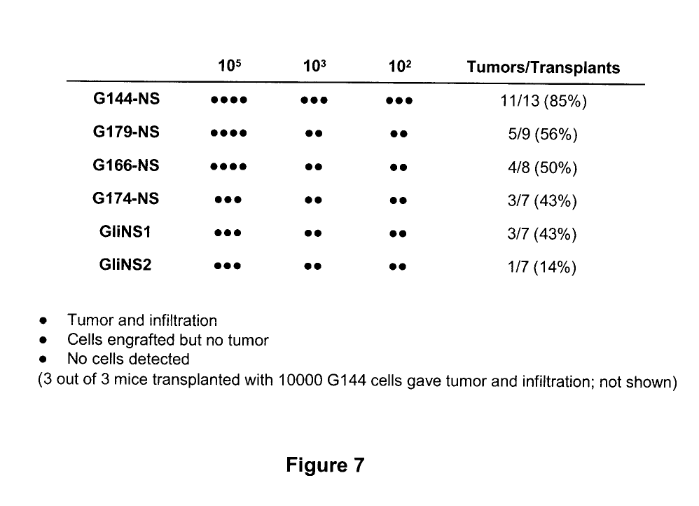

Xenotransplantation experiments were performed to determine whether the GNS cells would maintain the ability to induce tumors in a recipient animal. For these experiments, high numbers of G144-NS and GliNS1 cells were transplanted into the brain of a mouse. Five weeks following transplantation, the G144-NS and GliNS1 cells had survived and engrafted into the mouse brain (FIG. 6A-6D). Aggressive tumors formed in mice left for a further 15 weeks or more. The tumors observed were heavily vascularized and demonstrated features of glioblastoma multiforme by hemotoxylin and eosin staining. Analysis of the tumor mass by both histology and molecular markers reveals clear heterogeneity in the tumor. Comparison of the FACS quantitation of CD133 expression shows a reduction in immunopositive cells in the in the xenograft compared to the cell lines. Transplantation of 10⁵ cells resulted in a large tumor masses for all mice (n=7, 4/4 for G144-NS and 3/3 for GliNS1). Transplantation of five other cells lines also resulted in tumor formation, although not in every case (FIG. 7).

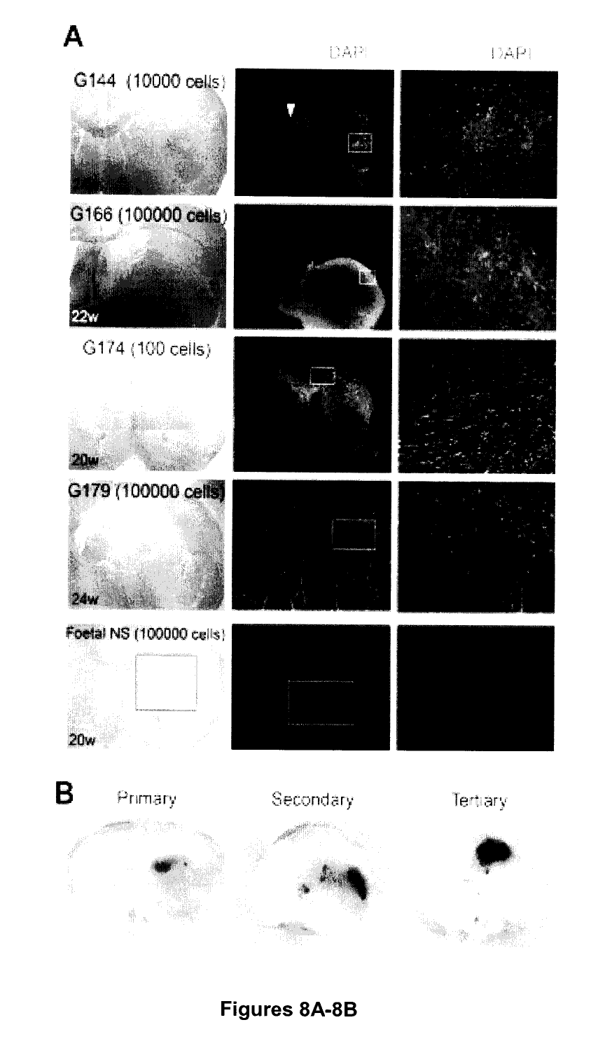

The number of cells required tumor formation upon transplantation in an animal is often indicative of the tumorigenicity of a cell. Although tumor formation was observed for each tested cell line, a reduced number of G174 and G144-NS cells were required to induce tumor formation compared to other tested cell lines (FIGS. 8A-8B).

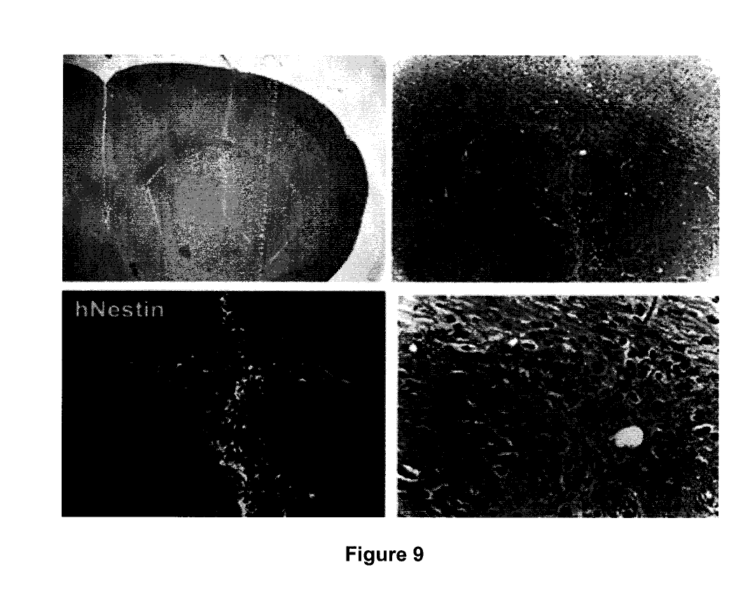

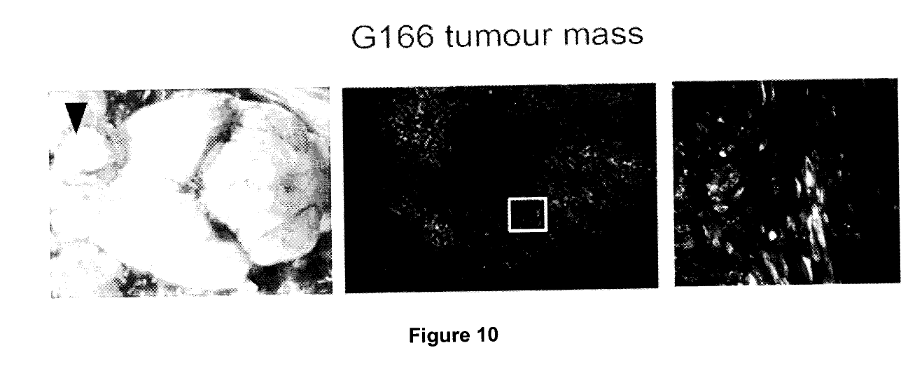

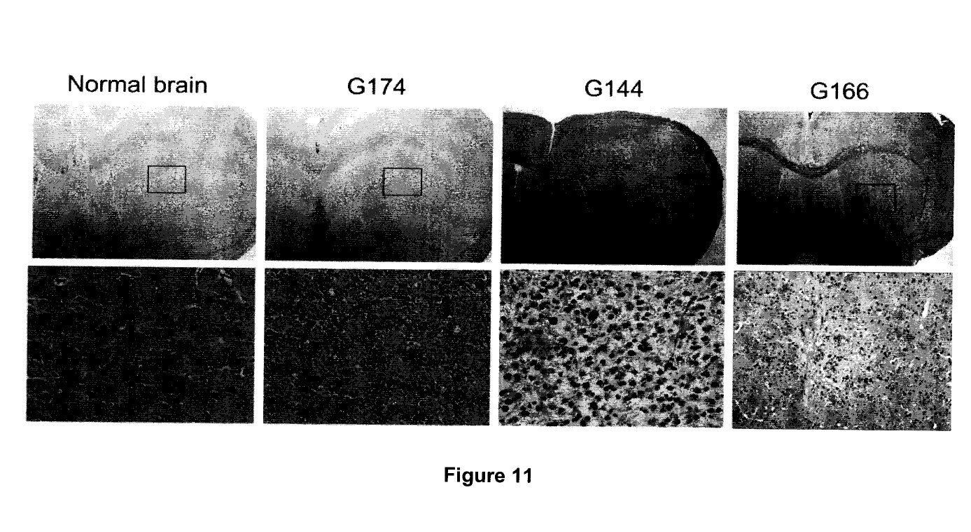

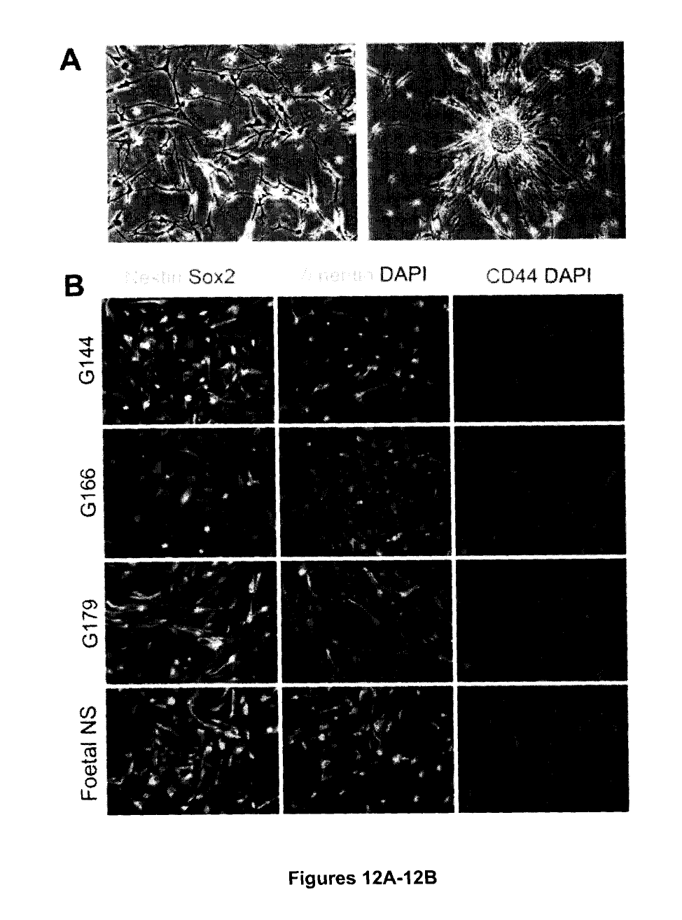

A number of the GNS cell lines demonstrate survival and engraftment following transplantation into an animal recipient (FIG. 9). The tumor that forms following transplantation of the GNS cell line often has the molecular and pathophysiological characteristics of the parent glioma from which the glioma neural stem cell was derived. For example, transplantation of G166-NS cells, derived from a giant cell glioblastoma tissue sample, results in the formation of a well-defined tumor mass with less infiltration into surrounding tissues (FIG. 10), a feature observed in patients with a giant cell glioblastoma. In addition, animal receiving transplanted G174-NS cells, G144-NS cells, and G166-NS cells also showed tumor formation (FIG. 11).

### Example 6

### Establishing Cell Lines from Human Gliomas

The key requirements for propagating both mouse and human NS cells without spontaneous differentiation or cell death are a combination of the growth factors EGF and FGF-2 on an adherent substrate (Conti et al., (2005) PLoS Biol 3, e283). We tested whether these conditions enable the isolation and expansion of stem cells from gliomas. Glioma tissue was recovered following surgical procedures and immediately processed, as described herein. Following direct plating onto a laminin-coated flask in NS cell culture media, we observed survival and establishment of primary cultures from all glioblastoma samples (FIG. 1A). There are a diversity of cellular phenotypes within these initial cultures, potentially reflecting mixtures of progenitors and differentiated cells, together with putative stem cells.

For some samples, high levels of cell death within the tumor mass interfered with establishment of adherent cultures, due to excessive cell debris binding to the substrate. In these instances we first plated cells in suspension culture where aggregates, or neurospheres, are formed. After 7-10 days, these were harvested free from dead cells and debris, and allowed to settle, attach, and outgrow on the substrate (FIG. 1A), as previously demonstrated for mouse and human NS cells (Conti et al., supra). Using each of these derivation approaches we are routinely able to generate adherent primary cultures of human malignant glioma cells.

To determine whether these primary glioma cell populations are expandable, we allowed cultures to grow to confluence and then began passaging cells continuously. Cultures had a doubling time of around 3-6 days and were typically split 1:3 or 1:4. Within 2-3 passages cultures appeared less heterogeneous. As for fetal NS cells, we find that a laminin substrate provides the most effective means to propagate the cells as monolayers, while parallel cultures grown on gelatin or untreated plastic undergo cell clumping, and cells detach (not shown). Using these adherent conditions we have been able to expand six cell lines for at least one year (>20 passages) without any obvious crisis or alteration in growth rate. Cell lines were established from histopathologically distinct types of tumor, namely: three cases of glioblastoma multiforme (G144, G166 Glioma NS cell lines and GliNS2), a giant cell glioblastoma (G179), and an anaplastic oligoastrocytoma (G174). Each line can be efficiently recovered following freezing and thawing. The cells are expanded in the absence of apoptosis, and can readily be genetically modified using nucleofection (not shown). To test the robustness of our protocol, for one glioma sample (Patient #144), we established cell lines independently in each of our laboratories using the same initial tumor sample. These cell lines were designated G144 and G144ED. In all subsequent analyses performed we have found no striking differences in behavior or marker expression between these two cell lines. Together these findings suggest that adherent NS cell culture conditions facilitate the routine establishment of cell lines from gliomas. Three cell lines (G144, G166, and G179) are characterized in detail in this study.

### Example 7

### Characterization of Glioma-Derived Cell Lines

To ascertain whether the glioma-derived cells have similarities to fetal NS cells (Sun et al., (2008) Mol Cell Neurosci 38, 245-258), we undertook a phenotypic characterisation of NS cell/neural progenitor cell markers. Immunocytochemistry confirmed that nearly all cells within the culture express Vimentin, Sox2, Nestin, and 3CB2, although for each of these there appears to be some variations in levels between cells (FIG. 1B and data not shown). Nuclear staining with DAPI reveals irregular nuclei and nuclear blebbing for G144. By time-lapse videomicroscopy, cells within G144 and G179 cultures display dynamic changes in cell shape, and are highly motile, both features of fetal NS cells. Quantitative data generated from cell tracking analysis showed that G166 cells are less motile than G144 cells, with the later moving on average 1.8 times further from their initial position. Thus, glioma-derived cell lines are broadly similar to normal NS cells, and thus termed glioma NS (GNS) cells.

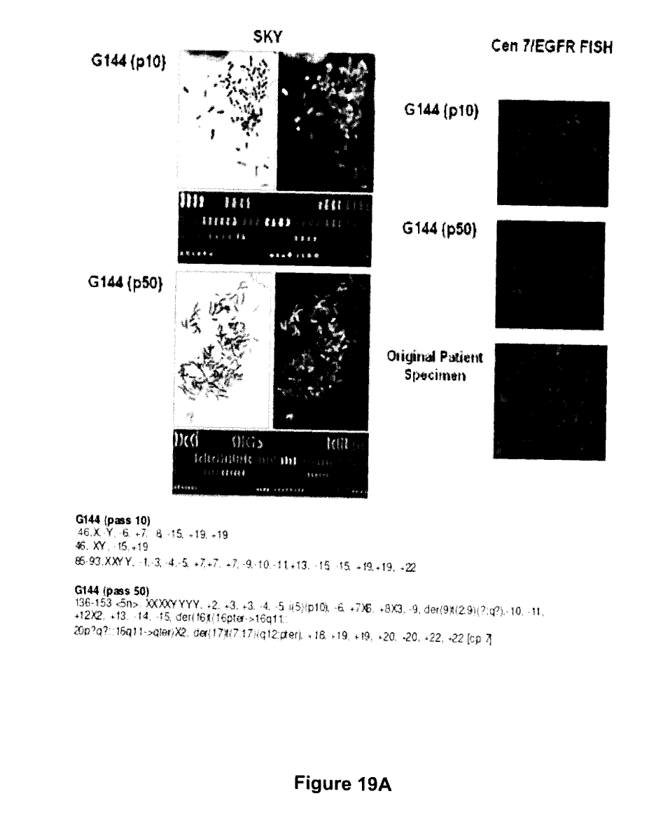

To determine whether GNS cells maintained chromosomal stability in culture, we performed molecular cytogenetic analyses using spectral karyotyping (SKY) and locus-specific FISH at early and late passages for G144 and G179 (FIGS. 19A-19B). Early G144 cultures exhibited no structural alterations, and contained a mixture of diploid (2n) and tetraploid (4n) cells. Simple clonal numerical gains of chromosomes 7 and 19; and losses of chromosomes 6, 8 and 15 were identified. By contrast, late passage G144 (passage 60) cultures exhibited a more complex and heterogeneous pattern of both numerical and structural chromosomal change; consistent with some loss of genome stability at higher passages. Numerical change involved multiple gains of chromosomal complements including both pentaploid (5n) and heptaploid (6n) cells. Acquisition of structural changes, included the clonal presence of an isochromosome 5(p) and the insertion of material from chromosome 20 into chromosome 16 as well as translocations was observed. A second population of cells contained additional rearrangements involving unbalanced translocations: der(17)t(7:17)(q12;pter) and der(9)t(2;9)(?;q?) was also seens. Simple low level clonal gains of multiple chromosomes were detected, but interestingly very high levels of polysomy of chromosome 7, with up to 14 copies in some cells, were evident. These numerical changes of chromosome 7 were confirmed by EGFR-specific interphase FISH using tissue samples derived from the surgical resection of the tumor, as well as cultured cells (FIG. 19A-19B). It is noteworthy that the EGFR, CDK6, and MET genes mapping to this chromosome are recurrently amplified and/or overexpressed in glioblastoma (Kleihues et al., (2000) Pathology and Genetics: Tumors of the Nervous System, 2nd Edition edn: IARC Press, Lyon). Similarly, G179 exhibited a more complex chromosomal pattern of numerical and structural change in later passage and like G144, polysomic gain of whole chromosome 7 was evident. In addition there was deletion of part of chromosome 10, containing the PTEN gene and an unbalanced translocations der(19)t(12;19)(q11;q11) generating 19q loss; der(21)t(13;21); and a der(22)t(17;22) generating a net gain of 17q. Collectively, the changes observed were consistent with a progressive loss of chromosomal stability in glioma derived cells at higher culture passage number. Moreover, the pattern of acquired numerical change in vitro has some parallels with the observed genomic alterations evident in patient tumors.

### Example 8

### Tumirogenicity of GNS Cells

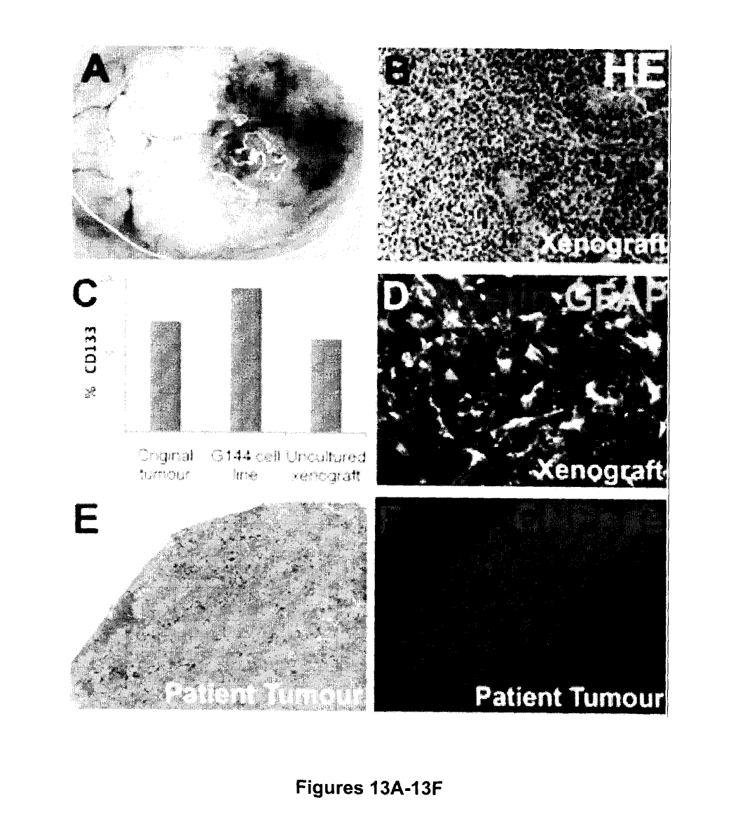

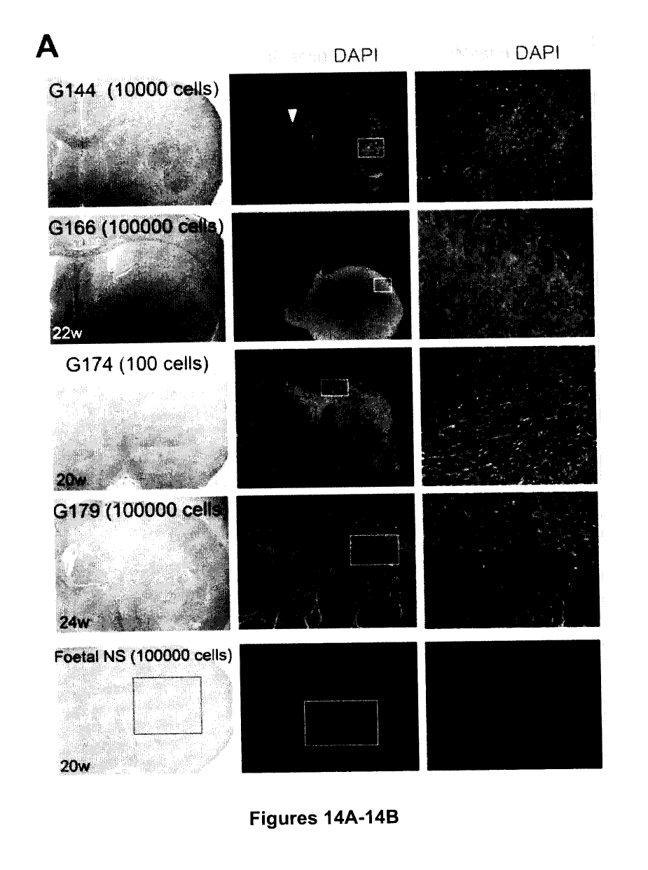

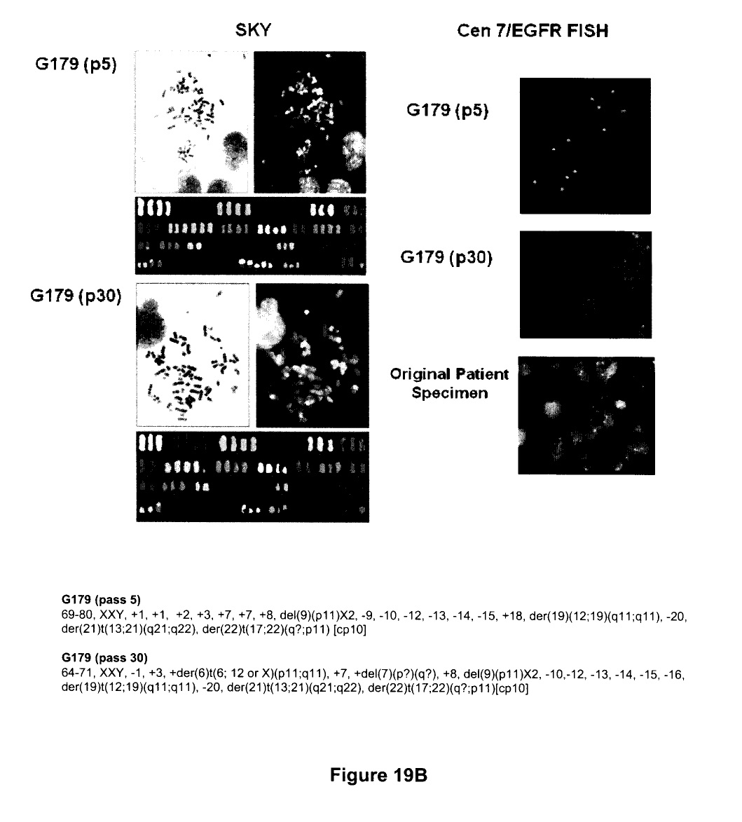

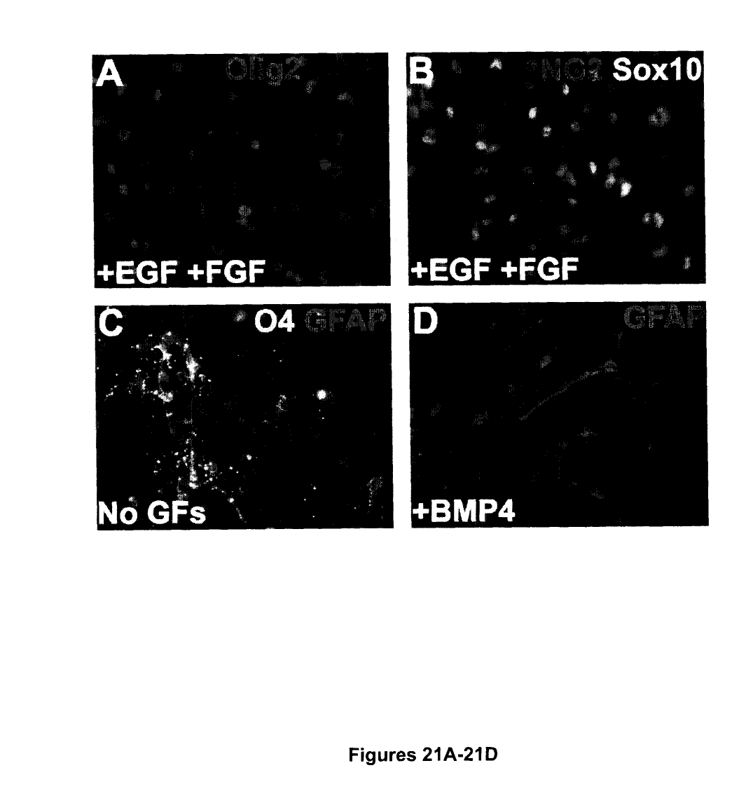

To test the capacity of GNS cells to initiate tumor formation, we carried out intracranial transplantation into immunocompromised mice. Initially we injected 100,000 cells from G144 cultures (expanded>10 passages). Five weeks later, a first cohort of mice was sacrificed, and we were able to identify large numbers of engrafted human nestin immunoreactive G144 cells that had infiltrated the host brain (FIGS. 20A-20D). A second cohort of mice was sacrificed after 20 weeks or longer. In these animals we typically observed formation of large and highly vascularised tumors (FIG. 13A). The histopathology of these xenograft tumors is strikingly similar to human GBM tumors, namely: pseudopalisading necrosis, nuclear pleomorphism, and extensive microvascular proliferation (Kleihues et al., (2000) Pathology and Genetics: Tumors of the Nervous System, 2nd Edition edn: IARC Press, Lyon) (FIGS. 13A and 13B). GNS cells differentiate in vivo and cellular heterogeneity is evident within the xenograft tumor population following immunostaining for Nestin and GFAP or flow cytometry for CD133 (FIGS. 13C and 13D). CD44 and Nestin-expressing tumor cells are frequently identified on the periphery of the tumors, with GFAP more prominent centrally, suggesting that the most primitive cells are invasive (FIG. 22A-22C). G166 and G179, as well as two other GNS cell lines tested (G174 and GliNS2) were also able to generate tumors (FIG. 14A and Table 2). Highly infiltrative behavior characterizes high-grade glioma, and makes full surgical resection of the tumor population impossible. In most transplants we saw a striking infiltration of the brain reminiscent of the human disease. An exception was G166, the CD133₋ cell line which generated a more defined tumor mass (FIG. 14A).

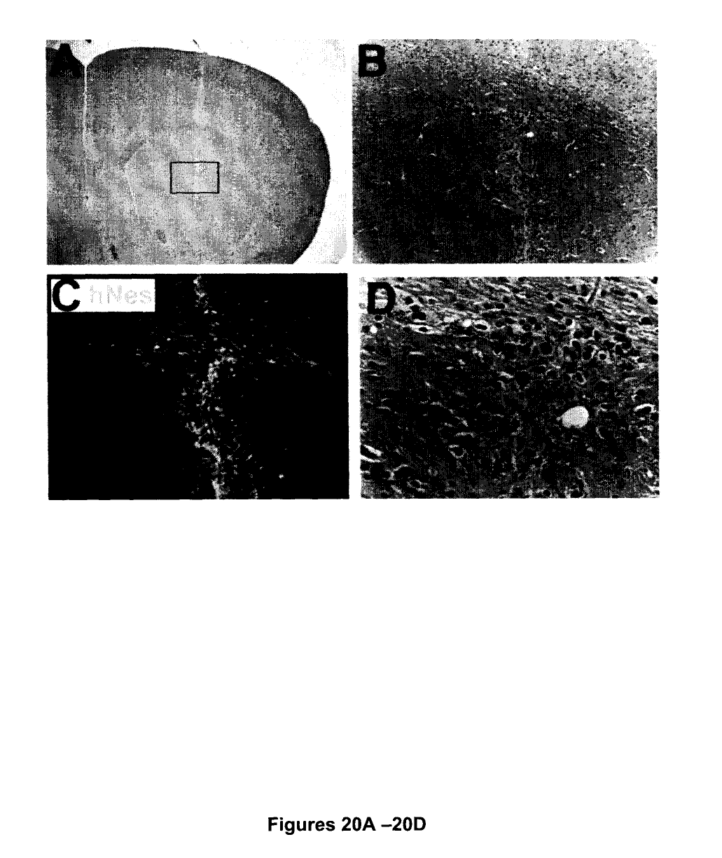

To calibrate tumor-initiating potency, we carried out transplantations using 10-fold dilutions of cells. The minimum number of cells tested (100), resulted in most cases in cell engraftment, and for two lines (G144 and G174) was sufficient to generate an aggressive tumor mass (Table 2). Clonal expansion from a single G144 cell in vitro followed by transplantation also resulted in similar tumors (FIGS. 21A-21D). These results contrast sharply with normal fetal NS cells, which never generated tumors even using 10⁵ cells (n=5) (FIG. 14A). To determine whether the tumor initiating cells self-renew within the xenograft, we carried out serial transplantations from the tumor mass into secondary and tertiary recipients using G144 cells. In each case, tumors were generated (FIG. 14B). Re-derivation of GNS cell lines from xenograft tumors was also straightforward using adherent conditions. However, this was less successful using suspension culture methods. Together, these data demonstrate that long-term expanded glioma derived stem cell lines remain highly tumorigenic, and are capable of forming tumors that appear to recapitulate the human disease.

TABLE 2

| | 10⁵ | 10³ | 10² |
|---|---|---|---|
| G144-NS | **** | *** | ••* |
| G144ED | *** | ♦• | ♦• |
| G166-NS | **•* | *♦ | •• |
| G179-NS | *♦** | *• | •♦ |
| G174-NS | *•* | •• | *• |
| GliNS2 | *•• | •• | •• |

*Tumor and infiltration
•Cells engrafted but no tumor
♦No cells detected
Five mice were injected with 10⁵ fetal NS cells (hf240) and no tumors formed

### Example 9

### Differentiation of GNS Cells

A defining property of stem cells is their ability to generate differentiated progeny. The most prevalent form of glioma is referred to as astrocytoma, based on the predominance of GFAP⁺ astrocyte-like cells within the tumor mass. However, GBMs also contain anaplastic cell populations, and in some cases an oligodendrocyte component (Kleihues et al., (2000) Pathology and Genetics: Tumors of the Nervous System, 2^{nd} Edition edn: IARC Press, Lyon).

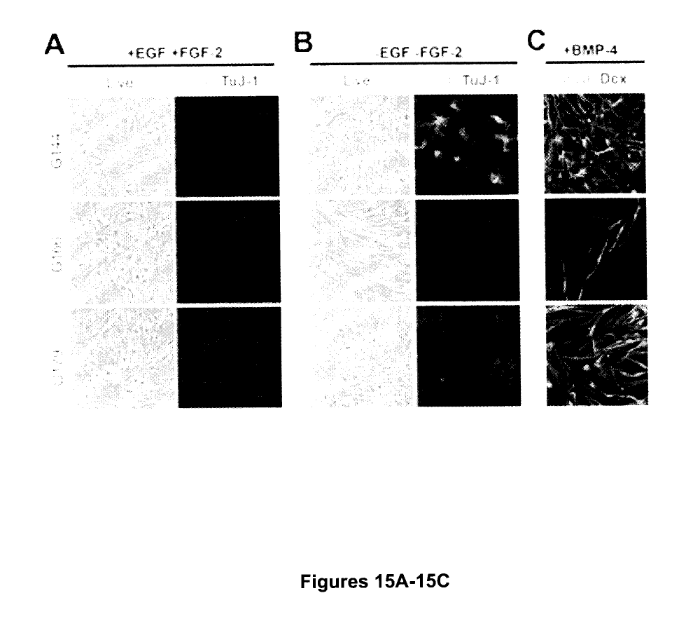

For all GNS cells analyzed, and in contrast to glioma neurospheres (Yuan et al., (2004). Oncogene 23, 9392-9400), we find differentiation to oligodendrocytes (O4⁺) or neurons (TuJ-1⁺) is fully suppressed in the presence of EGF and FGF-2 (FIG. 15A). We tested the capacity of GNS cells to undergo oligodendrocyte or neuronal differentiation upon growth factor withdrawal. In contrast to fetal NS cells, G144 and G179 GNS cells did not display elevated cell death in response to growth factor withdrawal and instead began to differentiate. For G144, we noted the appearance of significant numbers of O4⁺ or CNPase⁺ oligodendrocyte-like cells, within 1 week (FIG. 15B and data not shown). By contrast, G179 did not readily produce oligodendrocytes but mainly TuJ-1⁺ cells (FIG. 15B). Neuronal-like cells or oligodendrocytes were not apparent in G166 cultures, which continued to proliferate in the absence of EGF and FGF-2, suggesting autocrine/paracrine signaling or intrinsic signals are sufficient to drive self-renewal in this line (FIGS. 15A and 15B).

To determine whether GNS cells could respond to inductive signals and generate astrocytes, we exposed cells to BMP-4 or serum. For G144 and G179 within 7 days following addition of BMP-4, we observed a striking change in cell morphology and the majority of cells express high levels of GFAP, although in each case there was also a minor population of Doublecortin⁺ (Dcx⁺) neuronal-like cells (FIG. 4C). This response is similar to that of human fetal NS cells (Sun et al., (2008) Mol Cell Neurosci 38, 245-258). Similar results were seen using serum treatment. For G166, GFAP, cells could only be observed at low frequency following BMP treatment. Thus, while GNS cells retain a capacity to differentiate, the efficiency and lineage choice vary dramatically between each line.

### Example 10

### GNS Cells are Related to Specific Classes of Neural Progenitors

The ability of G144 cells to differentiate readily into oligodendrocytes upon withdrawal of growth factors was surprising. For mouse and human fetal NS cells, efficient oligodendrocyte differentiation requires a stepwise differentiation protocol involving exposure to exogenous signals, such as thyroid hormone, ascorbic acid, and PDGF, and results in heterogeneous populations of neurons, astrocytes and oligodendrocytes (Glaser et al., (2007) PLoS ONE 2, e298; Sun et al., (2008) Mol Cell Neurosci 38, 245-258). G144 cells may represent a corrupted tri-potent state that has acquired genetic changes that influence the lineage choice during differentiation, biasing towards oligodendrocyte commitment. Alternatively, G144 cells may have a distinct phenotype more similar to oligodendrocyte precursor cells (OLPs) than to NS cells. To distinguish between these two possibilities, we assessed established markers of OLPs (Olig2, Sox10, NG2, PDGFRα; reviewed in, (Zhang, S. C. (2001) Nat Rev Neurosci 2, 840-843), to identify whether they are expressed prior to or during differentiation.

Using immunocytochemistry we find that G144 cells, but not G166, G179, or human fetal NS cells, co-express Sox10 and NG2 in proliferating conditions, with the highest Sox10 expressing cells also expressing high NG2 (FIG. 5A). Quantitative RT-PCR confirmed that G144 cells express higher levels (>50-fold) of Olig2, PDGFRα and PDGFα, than other GNS cell lines and fetal NS cells. To verify that the observed marker heterogeneity was intrinsic to the GNS cells and not due to mixed populations we generated clonal cell lines and assessed marker expression. For each line (n=3), we saw heterogeneous expression of Olig2, Sox10, and NG2, similar to the parental line, and a capacity to generate oligodendrocytes upon growth factor withdrawal (Supplementary S3). G144 cells therefore stably exhibit an oligodendrocyte precursor-like phenotype, prior to initiation of differentiation by growth factor withdrawal. Consistent with the oligodendrocyte differentiation in vitro, histopathological examination of sections from G144 xenograft tumors, including those generated from G144 clonal lines, identified cells with the typical ‘fried-egg’ appearance indicative of an oligodendrocyte component (FIGS. 22A-22C). More significantly, although diagnosed as a malignant astrocytoma (GBM), re-examination of the original patient tumor for G144 also revealed a significant oligodendrocyte component based on histopathology and CNPase staining (FIG. 13F).

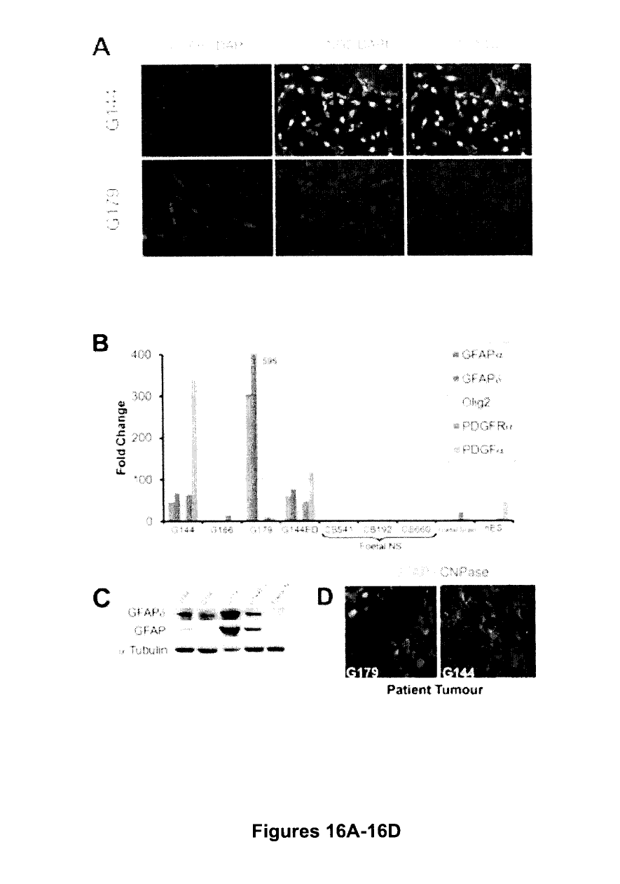

GFAP is expressed in radial progenitors/radial glia in the developing primate nervous system, as well as putative neural stem cells within the adult sub-ventricular zone (SVZ) (Doetsch et al., (1999) Cell 97, 703-716). Human fetal NS cell lines also express detectable levels of GFAP (Conti et al., (2005) PLoS Biol 3, e283). It is therefore not a specific marker of terminally differentiated astrocytes (Zhang, S. C. (2001) Nat Rev Neurosci 2, 840-843). Following BMP treatment, G179 cells expressed high levels of GFAP (FIG. 15C). However, we noted that even prior to treatment, G179 cells express detectable levels of GFAP, in contrast to G144 cells which are predominantly negative. To clarify the phenotype of G179 GFAP⁺ cells, we assessed levels of an alternative splice form of GFAP, termed GFAPδ, which has been shown to mark human SVZ astrocytes (Roelof et al., (2005) Glia 52, 289-300). Levels of expression of GFAPδ mRNA were >5 times higher in G179 than in G144 and G166 (FIG. 16B).

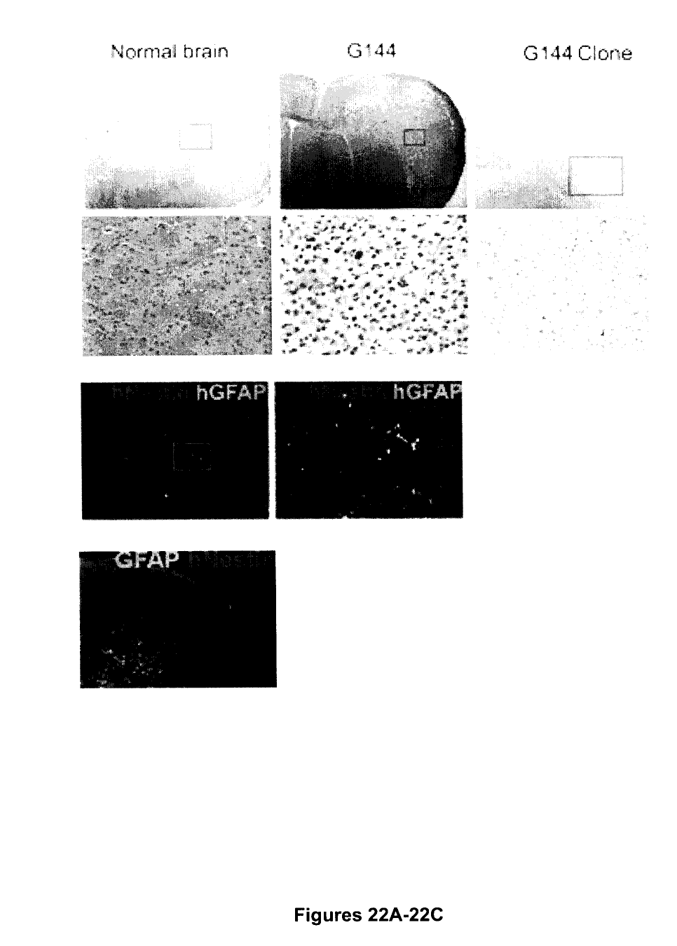

Immunoblot confirmed increased levels of protein (FIG. 16C), and by immunocytochemistry we could detect polymerized filaments of GFAPδ in G179 cultures (FIG. 16A). Co-expression of total GFAP and GFAPδ was confirmed by double staining (FIG. 23). We find peri-nuclear enrichment of GFAPδ filaments similar to the staining reported for SVZ astrocytes in vivo (Roelof et al., (2005) Glia 52, 289-300). Levels of GFAPδ drop significantly following in vitro differentiation (not shown). We also identified high GFAPδ-expressing cells within the original G179 patient tumor compared to G144, and these did not co-localize with CNPase-positive cells (FIG. 16D). The co-expression of GFAPδ, Sox2, Nestin and ability readily to generate neuronal-like cells in vitro, are features conserved with adult SVZ astrocytes (Jackson et al., (2006) Neuron 51, 187-199; Sanai et al., (2004) Nature 427, 740-744). G166 lacks expression of GFAPδ and OLP markers, but does express CD44 and can to some extent differentiate towards GFAP₊ astrocytes in vitro, which suggests some similarity to a more restricted astrocyte precursor. Together these findings suggest that despite their shared capacity to proliferate in response to EGF and FGF-2 and the widespread expression of neural progenitor markers, there are underlying differences between GNS cell lines. This may reflect their relatedness to distinct subtypes of ‘normal’ neural progenitor. These data further suggest that GFAPδ may be of use in identifying astrocyte-like cells that have stem cell properties.

### Example 11

### Global mRNA Gene Expression Patterns in GNS Cells

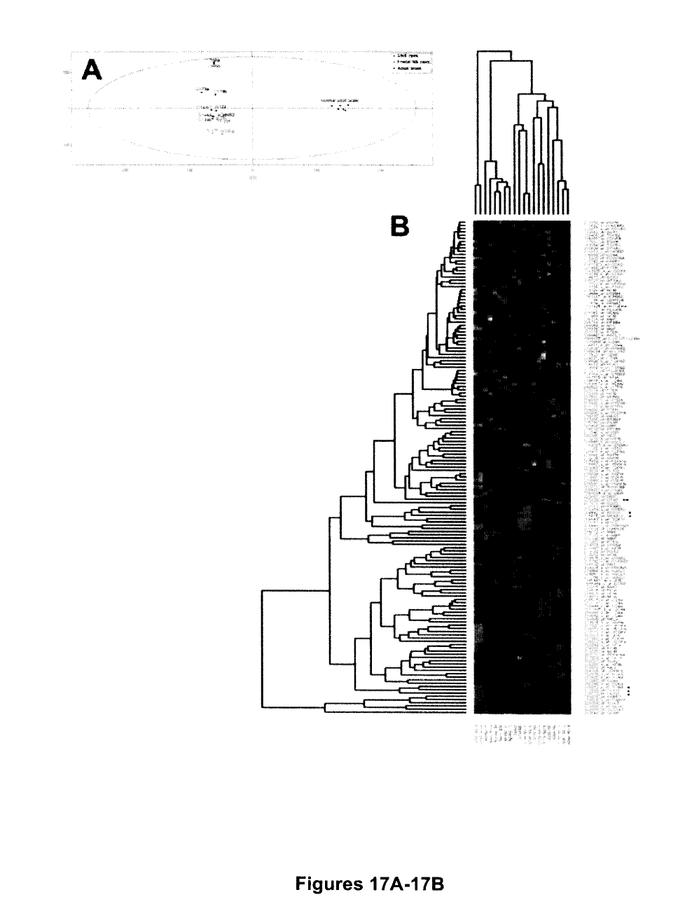

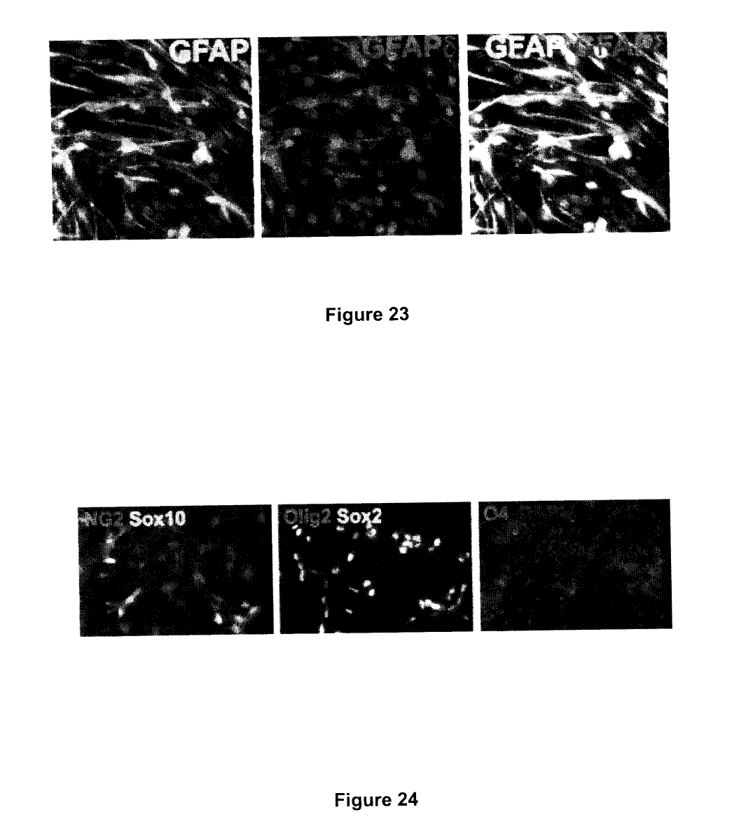

To evaluate the relationship between each GNS cell line and their correspondence to fetal NS cells, we carried out global mRNA expression profiling using microarrays. Principal component analysis revealed that each GNS cell line has a transcriptional state more closely related to fetal NS cells than adult brain tissue (FIG. 6A). Encouragingly, G144 and G144ED, the two lines established in independent laboratories from the same initial tumor sample, cluster together. This suggests that the observed tumor-specific differences between lines are not simply a reflection of selective events in culture. Consistent with our initial marker analysis, we find G179 and G166 express a distinct expression profile, both from one another, and to G174, G144, and GliNS2. To confirm the differential expression of markers between each GNS line, we performed a cluster analysis using known NS cell and lineage-specific markers, as well as pathways known to be disrupted in gliomas (FIG. 17B). The dendrogram generated is similar to that for the global PCA analysis, indicating that this set of markers is sufficient to distinguish between lines. These data also confirmed that G144 expresses the OLP cell markers Sox8, Sox10, Olig1, Olig2, Nk×2.2, while these are down-regulated in G179, which has higher levels of GFAPδ. GliNS2 clusters closely with G144 and also expresses the OLP cell markers suggesting that the phenotype of G144 may not be unique (FIG. 17B). Indeed, we confirmed using immunostaining that GliNS2 expresses NG2, Olig2, and Sox10, and can generate oligodendrocytes readily upon growth factor withdrawal (FIG. 24). We found no evidence for expression of the pluripotency markers Oct4 or Nanog in any of the samples.

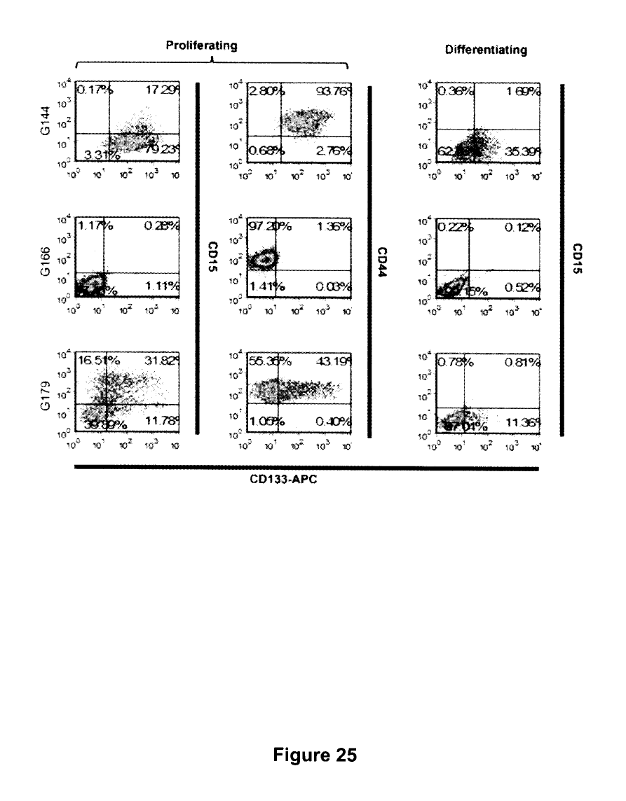

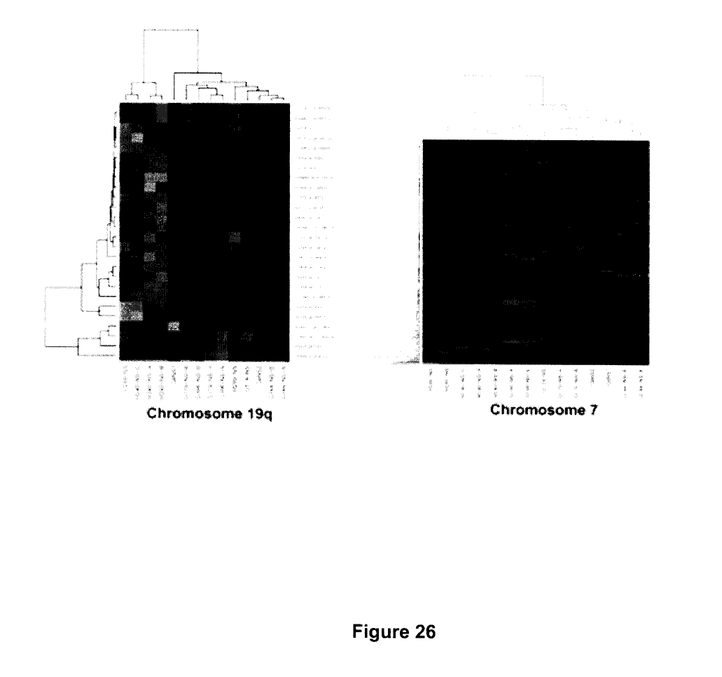

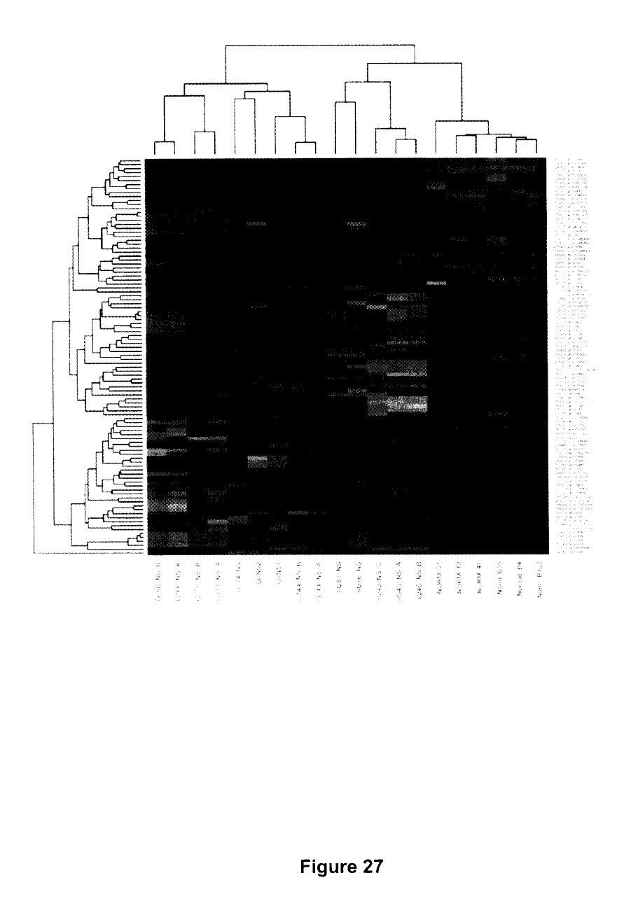

G166 expresses higher levels of EGFR than any other line, perhaps contributing to its resistance to differentiation upon EGF withdrawal or BMP treatment. We also noted an apparent lack of mRNA for prominin-1 (CD133). Using flow cytometry, we examined the status of the cell surface markers CD133 and CD15/SSEA-1, which mark fetal and adult neural progenitors (Capela et al., (2002) Neuron 35, 865-875), and also brain tumor initiating cells (Singh et al., (2004) Nature 432, 396-401). For G144 and G179, we observe an underlying heterogeneity within GNS cell cultures, similar to fetal NS cells, while G166 is negative consistent with the low mRNA expression (FIG. 25). We also found no evidence for CD133 expression within the original G166 tumor sample (not shown). By contrast, we find that for each glioma line, including G166, the hyaluronic acid binding protein, CD44, is uniformly expressed. CD44 has previously been characterised as an astrocyte precursor marker, but we recently demonstrated that it also marks NS cells in vitro (Pollard et al., (2006) Cerebral Cortex, 16 Suppl 1, i112-20). To identify new candidate markers that distinguish fetal NS cells from GNS cells, we identified the most significantly differentially expressed transcripts across all six GNS cell lines versus three fetal NS cells. Genes located on chromosome 7 were significantly overrepresented within this set (FIG. 26). This was not unexpected given the variable copy number increases for this chromosome seen by SKY (FIGS. 19A-19B). Perhaps more surprising was the identification of reduced expression in GNS cells for genes located on chromosome region 19q. While this region is frequently deleted in oligoastrocytoma and secondary GBM, it is a less common feature of primary GBMs (Kraus et al., (1995) J Neuropathol Exp Neurol 54, 91-95; Nakamura et al., (2000) J Neuropathol Exp Neurol 59, 539-543; Reifenberger et al., (1994) Am J Pathol 145, 1175-1190). The set of top 100 differentially expressed genes (excluding those on chromosome 7 or 19q) provides a set of candidate markers that distinguish fetal NS cells from GNS cells (FIG. 27). The most significantly down-regulated gene in GNS cells relative to fetal NS cells is the well studied tumor suppressor PTEN which is often lost or mutated in gliomas and other cancers (Louis, (2006) Ann Rev Pathol 1, 97-117).

### Example 12

### Drug Screening Using GNS Cells

The mouse neurosphere culture system has proved useful for screening of compounds that affect neural stem cell expansion, using growth assays (MTT incorporation) (Diamandis et al., (2007) Nat Chem Biol 3, 268-273). However, there are several inherent limitations of this system for application in high-throughput drug screening. Firstly, human neural stem cells expand more slowly in vitro than their mouse counterparts, and this means that accurate assays quantifying cell proliferation are required for rapid screening. This is difficult using suspension cultures due to extensive cell death. Secondly, the neurosphere population also includes restricted progenitors and differentiated cell types and it is therefore difficult to identify the precise cellular target, as real-time monitoring of cell behavior is not possible. Finally, fusion of neurospheres commonly occurs in suspension, which confounds quantitative analyses based solely on sphere numbers or size (Singec et al., (2006) Nat Methods 3, 801-806). Many of these hurdles are overcome using monolayer GNS cells. Therefore, we carried out a chemical screen using a live-cell imaging system (IncucyteHD) to monitor the effects on GNS cell behavior of 450 compounds (NIH Clinical Collection). This collection comprises known drugs that have passed phase I-III trials and have been used in the clinic. Drug re-profiling/repositioning (i.e., the new application of drugs already at market) bypasses the time and cost constraints associated with new drug development, and should result in rapid translation of basic findings to the clinic (Chong et al., (2007) Nature 448, 645-646). Following addition of 10 μM of each drug we simultaneously captured live images of each well at 30 min intervals over a two day period (six parallel 96-well plates). The relative change in cell number within each individual well was determined at each timepoint.

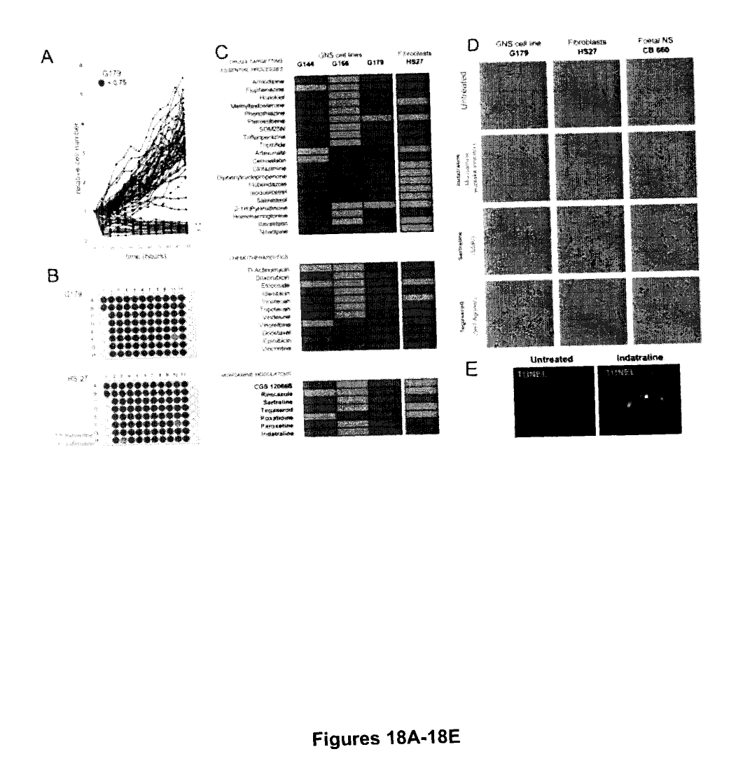

We carried out two fully independent screens using G144, G166, and G179, as well as a human fibroblast cell line (HS27). We were able to identify 38 drugs that had clear cytotoxic or cytostatic effects on at least one line (FIGS. 18A-18C). Images captured for each of these wells were used to generate time-lapse movies of cell behaviour following drug treatment, and visual inspection of these confirmed clear effects of each compound. Predictably, included within this set were drugs that disrupt core cell biological processes, including anthracycline chemotherapeutics (doxorubicin and idarubicin), the anti-mitotic vindesine, and DNA topoisomerase inhibitors (irinotecan and etoposide) (FIG. 18C). However, we also found line-specific effects for 15 of the ‘hits’ consistent with the individualized phenotypes of GNS cells (FIG. 18C). Our previous studies had identified a sensitivity of mouse neurospheres to alterations in neurotransmitter signaling pathways (Diamandis et al., (2007) Nat Chem Biol 3, 268-273). Intriguingly, of the 23 drugs that killed all GNS cell lines, seven are known to modulate the monoamine signalling pathways. Three are monoamine reuptake inhibitors (indatraline and paroxetine), a serotonin-specific reuptake inhibitor (sertraline), two serotonin receptor agonists (CGS 12066B and tegaserod), two dopamine receptor antagonists (10H-phenothiazine and Trifluoperazine) and a dopamine transporter/sigma receptor modulator (Rimcazole). A monoamine oxidase inhibitor (tryptoline) was also seen to have an effect, although this was initially excluded using thresholds set for growth rates. For indatraline and paroxetine, we saw no effect on fibroblasts. We chose Glioma NS cell lines to validate several of the drugs, from each class, using compounds obtained from an independent supplier (FIG. 18D). We also showed that cells were dying by an apoptotic pathway when treated with indatraline (FIG. 18E).

The addition of indatraline, rimcazole, or sertraline, resulted in cell death for all tumor lines and fetal NS cells, but had less striking or no effect on the fibroblast cells. Taken together, these results highlight the utility and scalability of adherent GNS cell lines for high-throughput drug screening, and extend our previous findings suggesting that brain cancer stem cells may be acutely sensitive to modulation of monoamine signaling, and particularly, the serotonin signaling pathway.

### Example 13

### Experimental Methods

These methods were used to generate the experiments described above.

Glioma Primary Cell Cultures

Brain tumor samples were obtained from patients treated at hospitals in Toronto and Edinburgh area following local ethical board approval. G144 and G144ED (51 yr. male), G166 (74 yr. female), and GliNS2 (54 yr. male), were all diagnosed as classic glioblastoma muliforme (GBM). G179 (56 yr. male) was a GBM (giant cell variant). G174 (60 yr male) was an anaplastic oligodendroglioma). Tumor samples were collected in PBS placed on ice and typically processed within 30-60 min. For those samples of poor quality, we first micro-dissected the tumor to remove regions of necrosis and blood vessels prior to enzyme based cell dissociation. Tumors were dissociated into single cells by placing in Accutase (Sigma) for 15-20 min at 37° C. and then triturated (Edinburgh), or using previously using the enzyme cocktail previously described (Toronto) (Singh et al., 2003). Cell suspensions were then passed through 50 μM cell strainer and plated into NS cell media. For those tumors with excess debris, cells were initially allowed to form spheres/aggregates in suspension culture, and these were then transferred to a fresh laminin-coated flask. They subsequently attached and began to outgrow over the course of a week.

Expansion of GNS Cells

GNS cell expansion was carried out as described previously for human foetal NS cells (Sun et al., (2008) Mol Cell Neurosci 38, 245-258). Tissue culture flasks were pre-treated with Laminin, 10 μg/ml in PBS, (Sigma), for at least 3 hrs at 37° C. GNS expansion media comprised Euromed-N media (Euroclone) supplemented with modified N2 supplement (in house preparation as described in (Pollard et al., (2006) Methods Enzymol 418, 151-169), plus 1× B27 (Gibco). For more recent experiments cells were expanded using RHB-A Neural differentiation media (Stem Cell Sciences) or Neurocult-Human media (Stem cell technologies). Each of these basal media was supplemented with the growth factors EGF and FGF-2 20 ng/ml of each (Peprotech), plus heparin (2 μg/ml). As for human fetal NS cells, we find that the cytokine LIF had no apparent effect on the cells. GNS cells were routinely grown to confluence, dissociated using Accutase (Sigma), and then split 1:3 to 1:5. Media was replaced with fresh media every 3-5 days. For all routine analysis we typically worked with cells between passage 10 and 20. For freezing, we re-suspended cell pellets in 0.5 ml of 10% DMSO/Media and placed in a −80° C. freezer. For long-term storage, liquid nitrogen was used. Cells demonstrated only minimal cell death upon thawing. Fetal NS cells CB541 and CB660 are described by Sun et al., 2008, supra), while hf240, hf286, and hf289 (used in the microarrays) were isolated using similar techniques.

Spectral Karyotyping (SKY)

Mitotically active cultures were colcemid treated and prepared for cytogenetic harvest (Bayani et al., (2004) Current protocols in cell biology, Chapter 22, Unit 22 22.). Spectral Karyotyping (SKY) was performed using the commercially available kit provided by Applied Spectral Imaging (Vista, Calif.) according to the manufacturer's instructions. The slides were imaged and analyzed fluorescent microscope (Carl Ziess Canada) and the imaging software provided by ASI.

Fluorescence In Situ Hybridization (FISH)

FISH was performed on either cytogenetic preparations or formalin-fixed paraffin embedded (FFPE) sections using the commercially available Centromere 7 and EGFRlocus specific FISH probes provided by Vysis (Abbott Technologies). For cytogenetic preparations, the probe was applied and slide processed according to the manufacturer's instructions. For FFPE sections, the 5 μm tissues were dewaxed and dehydrated. Following a 1 hr incubation in 10 mM sodium citrate (pH=6.0) at 80° C., the slides were pepsin treated. After a final dehydration, the probe was applied to the slide and co-denatured for 10 min at 78° C. and allowed to hybridize overnight. Posthybridization washes were performed according to the manufacturer's instructions and slides were counterstained with DAPI in an antifade solution.

Differentiation of GNS Cells

All differentiation was carried out on laminin-coated plastic, either in 4-well plates (˜0.5-1×10⁵ cells/well) (Nunc), or for time-lapse movies using 24-well Imagelock microplates (Essen Instruments). For oligodendrocytes and neuronal differentiation we used the same basal media but lacking EGF or FGF-2 (i.e., growth factor withdrawal). For astrocyte differentiation, we supplemented basal media with either BMP at 10 ng/ml (R and D systems), or 1% serum (Sigma). In each case, cells were washed twice with PBS or minimal media before adding the final differentiation media. Samples were processed for immunocytochemistry, typically 7-10 days later.

### Immunocytochemistry

Cells w ere fixed in 4% PFA for 10 min and then washed with PBS+0.1% TritonX-100 (PBST). Blocking was carried out using 1% goat serum for 30 mins. Primary antibodies were incubated overnight at 4° C.; secondary antibodies for 1 hr at room temperature. Primary antibodies: human Nestin, (1:500), O4 (1:100, live stain), Sox2 (1:50), (R&D systems); Vimentin (1:50), 3CB2 (1:20), (DSHB, Univ. of Iowa), TuJ-1 (1:500) (Covance), CD44 (1:100, live stain) (E-bioscience); GFAP (1:300) (Sigma, monoclonal GA-5); NG2 (1:100), Olig2 (1:200), GFAP [symbol] (1:200), (Chemicon). We used a goat secondary antibody conjugated to Alexa dyes, 1:1000 (Molecular Probes). DAPI was used as nuclear counterstain (Sigma). Images were acquired using a Leica DMI400B inverted fluorescence microscope linked to a DFC340FX camera.

Flow Cytometry

CD133 (1:5) (Miltenyi); CD15 (1:100) (BD); CD44-PE/Cy5 (1:1000) (eBioscience) were used for flow cytometry. Clonal cell lines were established using flow cytometry (MoFlo, Dako) to deposit single cells into each well of a 96-well plate.

Mouse Brain Fixation, Histopathology, and Immunohistochemistry

These procedures were carried out as described previously (Singh et al., 2004). Antibody staining was carried out following deparaffinization and heat induced antigen retrieval using citrate buffer (pH 6.0). The antibodies used were CNPase 1:200 (Sigma), hNestin 1:200 (Millipore), hGFA P 1:200 (Sternberger monoclonals), GFAP [symbol] 1:500 (Millipore).

Xenotransplantation

GNS cells were injected stereotactically into 6- to 8-week-old NOD-SCID mouse frontal cortex, following administration of general anaesthesia. The injection coordinates were 3 mm to the right of the midline, 2 mm anterior to the coronal suture and 3 mm deep.

Microarrays and Bioinformatics

All expression profiling was carried out using the GeneChip® Human Genome U133 Plus 2.0 Array (Affymetrix). Data were pre-processed using various Bioconductor packages: affyQCReport for quality control checks and the vsnrma function of the Bioconductor package vsn for data normalisation. The limma package in Bioconductor was used to statistically analyze the data using both the modified t-test and F-test and the false discovery rate (FDR) method for multiple hypothesis correction. To compare the three different condition groups: ‘brain,’ ‘fetal,’ and ‘glioma’, a general significance threshold of p<0.05 was taken for each comparison. Dendograms and heatmap plots were created using the hclust package in Bioconductor software. Hierarchical clustering (using the Euclidean distance and the average linkage method) was performed on the normalized data set and then on various lists of statistically significant differentially expressed genes. The Umetrics software was used to perform a principal components analysis (PCA) on the normalised data set and partial least square discriminant analysis (PLS-DA) was used to determine group classifiers.

The web-based tool, GeneTrail (http://genetrail.bioinfuni-sb.de/) (Backes et al., (2007) Nucleic Acids Res 35, W186-192.), was used to perform both a over-representation analysis (ORA) and a gene set enrichment analysis (GSEA) on the 1663 genes found to be statistically significant (P<0.05) when comparing ‘glioma’ versus ‘fetal’ sample groups.

Real-Time PCR

Total mRNA was harvested using the Qiagen RNeasy kit (Qiagen). cDNA was generated using Superscript III (Invitrogen) and quantitative PCR carried out using the LightCycler system (Roche). All PCRs are a mean of biological and technical duplicates. Samples were normalized using beta-actin primers and the data presented is normalized to sample fetal NS cell (CB660). Primers were designed using Primer 3 software (MIT), and had the following sequence:

| | |
|---|---|
| GFAPdeltaF | ACATCGAGATCGCCACCTAC, |
| GFA PdeltaR | CGGCGTTCCATTTACAATCT, |
| GFA PalphaF | ACATCGAGATCGCCACCTAC, |
| GFAPalphaR | ATCTCCACGGTCTTCACCAC, |
| PDGFRaF | CCACCGTCAAAGGAAAGAAG, |
| PDGFRaR | CCAATTTGATGGATGGGACT, |
| PDGFaF | GATACCTCGCCCATGTTCTG, |
| PDGFaR | CAGGCTGGTGTCCAAAGAAT, |
| Olig2F | CAGAAGCGCTGATGG, |
| Olig2R | TCGGCAGTTTTGGGT. |

Immunoblotting

A 10% protein gel (Invitrogen) was used, and blotting was performed using the iBlot Dry Blotting system (Invitrogen). Antibodies used were: anti alpha-tubulin antibody at 1:5000 (Abcam), anti GFA Pdelta 1:500 (Chemicon), and GFA P 1:500 (Sigma). Secondary antibody conjugated to HRP were used with the ECL system to detect protein (Amersham).

Timelapse Movies and Drug Screening

For routine time-lapse imaging and generation of growth curves, we used the Incucyte system (Essen Instruments, USA). For cell tracking analysis we processed image stacks using ImageJ and analyzed cell tracks using the MTrackJ Plugin (http://rsb.info.nih.gov/ij/).

For the drug screen we used the IncucyteHD system (Essen Instruments, USA), which enables simultaneously monitoring of six 96-well microplates. GNS cell lines were plated at 10-20% confluence on 96 well plates (Iwaki) coated with laminin (10 μg/ml for 3 hours). The NCC NIH Chemical Compounds library (http://www.nihclinicalcollection.com/) was added to the plates at a final concentration of 10 μM per compound per well (DMSO 0.1%). Images were captured before and after the addition of the library every half hour for 2.5 days in an automated manner using the Incucyte HD device (Essen Instruments, USA). Relative increase in cell number values were generated for every well using confluence readings obtained at each time-point relative to the starting confluence. For every cell line (G144, G166, G179, and HS27) two independent screens were run. HS27 is a human foreskin fibroblast line (American Type Culture Collection). Cell number variation ranged from 2 to 4 fold within the 5* and the 95* percentile and showed a marked drop within the 5* percentile containing drugs potentially resulting in cell death. Every well associated with a reduction in cell number within the 5* percentile in at least 3 independent screens was visually inspected. The Z-factor for the screen was 0.76, indicating “an excellent assay” (Zhang et al., 1999). For validation, a few chosen compounds were received from an independent supplier Indatraline, Rimcazole, Sertraline (Sigma), Tegaserod (Sequoia Research Products) and a similar set of experiments were conducted on a lower scale on 24 wells with 2 μM or 10 μM over the same period of time.

Deposit Information

The neural tumor cell lines G179-NS and G144-NS were each deposited under Accession Numbers PTA-8894 and PTA-8895 under the Budapest Treaty, respectively at the American Type Culture Collection (ATCC), 10801 University Blvd., Manassas, Va. 20110-2209, USA on Jan. 23, 2008. Viability of each cell line was tested on Feb. 25, 2008, and the cultures were found viable.

### Other Embodiments

All patents, patent applications, and publications mentioned in this specification are herein incorporated by reference to the same extent as if each independent patent, patent application, or publication was specifically and individually indicated to be incorporated by reference.

1. A neural tumor stem cell which expresses at least one protein selected from the group consisting of nestin, Sox2, vimentin, CD44, CD15, CD133, GFAP, GFAPδ, and NG2 and has the ability to propagate in an in vitro culture.

2. The cell of claim 1, wherein said tumor is glioblastoma multiforme, giant cell glioblastoma, oligodendroglioma, ependymoma, or medulloblastoma.

3. The cell of claim 1, wherein said cell is capable of differentiating into neural cell types or is capable of inducing tumor formation when transplanted into the brain of a mammal.

4. (canceled)

5. The cell of claim 1, which can be propagated in culture for at least 20 passages.

6. The cell of claim 1, wherein said cell expresses at least two proteins selected from the group consisting of nestin, Sox2, vimentin, CD44, CD15, CD133, GFAP, GFAH, and NG2 or expresses Sox2, Nestin, CD44, and CD15.

7. (canceled)

8. The cell of claim 1, wherein the cell is a human cell.

9. A cell of claim 1, wherein said cell is from the cell line G144-NS (ATCC Deposit No. PTA-8895), G166-NS, G174-NS, G179-NS (ATCC Deposit No. PTA-8894), GliNS1, GliNS2, or EP253-NS.

10-15. (canceled)

16. A method of producing a neural tumor stem cell line, said method comprising the steps of:
(a) providing a neural tumor sample;
(b) culturing cells from said tumor sample under conditions which induce formation of neural cell spheres;
(c) dissociating cells from said spheres;
(d) applying said cells of step (c) to a substrate under conditions which allow adherence of said cells; and
(e) culturing said cells of step (d), thereby generating a neural tumor stem cell line.

17. The method of claim 16, wherein said substrate is charge-modified polystyrene or is poly-L-ornithine/laminin treated polystyrene.

18. (canceled)

19. A neural tumor cell line produced by the method of claim 16.

20. A method of identifying a candidate compound for the treatment of a neural tumor, said method comprising the steps of:
(a) contacting a neural tumor stem cell capable of undergoing proliferation with a compound; and
(b) measuring cellular proliferation of the tumor stem cell following treatment with said compound, wherein a compound that reduces proliferation of said cell, as compared to in the absence of said compound, is identified as a candidate compound for the treatment of a neural tumor.

21. The method of claim 20, wherein said candidate compound is selected from a chemical library.

22-24. (canceled)

25. The method of claim 20, wherein said cell is from a cell line selected from the group consisting of G144-NS, G166-NS, G174-NS, G179-NS, GliNS1, GliNS2, and EP253-NS.

26. A method of producing an animal model of a neural tumor comprising the steps of:
(a) providing at least one neural stem tumor cell, and
(b) transplanting said at least one cell into a nervous tissue of a recipient animal.

27. The method of claim 26, wherein said animal is a rodent or said cell is a human cell.

28. (canceled)

29. The method of claim 26, wherein the neural tumor cell is a glioma neural stem cell.

30. The method of claim 29, wherein said GNS cell is from a cell line selected from the group consisting of G144-NS (ATCC Deposit No. PTA-8895), G166-NS, G174-NS, G179-NS (ATCC Deposit No. PTA-8894), GliNS1, GliNS2, and EP253-NS.

31. (canceled)

32. A method for determining whether to administer a compound to a patient having a neural tumor, said method comprising the steps of:
(a) providing a cell from neural tumor stem cell line, wherein said stem cell line is derived from a neural tumor cell cultured under conditions sufficient to generate said cell line;
(b) contacting said cell from said cell line with said compound; and
(c) measuring the proliferation or viability of said cell, wherein a therapeutic agent that reduces proliferation or viability of said cell is identified as a potential therapeutic agent for said patient.

33. The method of claim 32, wherein said contacting step (c) further comprising contacting a second therapeutic agent.

34. The method of claim 32, wherein neural tumor cell is from a human, said compound is from a chemical library, or said compound is a chemotherapeutic agent.

35-36. (canceled)
